(19) United States
(12) Patent Application Publication
(10) Pub. No.: US 2020/0215036 A1
(43) Pub. Date: Jul. 9, 2020

(54) BENZIMIDAZOLE COMPOUNDS AS KINASE INHIBITORS

(71) Applicant: GB005, Inc., San Diego, CA (US)

(72) Inventors: Alain Laurent, Lachine (CA); Stephen J. Morris, Beaconsfield (CA)

(21) Appl. No.: 16/641,199

(22) PCT Filed: Aug. 21, 2018
(86) PCT No.: PCT/US2018/047353

§ 371 Date: Feb. 21, 2020

(30) Foreign Application Priority Data
Aug. 21, 2017 (CA) 2976819

(51) Int. Cl. A61K 31/422; A61K 45/06; A61K 31/5377; A61K 31/496; C07D 413/14

(52) U.S. Cl. CPC A61K 31/422; A61K 45/06; C07D 413/14; A61K 31/496; A61K 31/5377

(57) ABSTRACT

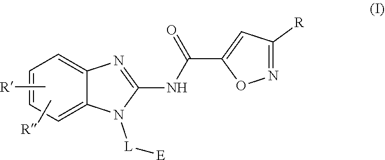

The present invention relates to a novel family of covalent kinases inhibitors. Compounds of this class have been found to have inhibitory activity against members of the TEC kinase family, particularly RLK (TXK). The present invention is directed to a compound of Formula (I) or pharmaceutically acceptable salt, solvate, solvates of salt, stereoisomer, tautomer, isotope, prodrug, complex or biologically active metabolite thereof, for use in therapy.

### BACKGROUND

### Technical Field

The present invention relates to a novel family of protein kinase inhibitors, their pharmaceutically acceptable salts, to pharmacological compositions that contain them and to their use of the inhibitors to treat or prevent diseases, disorders and conditions associated with kinase function.

### Description of the Related Art

Protein kinases are a large group of intracellular and transmembrane signaling proteins in eukaryotic cells (Manning G. et al, Science, 2002, 298: 1912-1934). Phosphorylation of specific amino acid residues in target proteins by protein kinases can modulate their activity leading to profound changes in cellular signaling and metabolism. Kinases play key roles in the regulation of cellular proliferation, survival, differentiation and function. Many kinases have been implicated in disease and, as such, are attractive therapeutic targets.

The Tec-kinase-family of kinases consists of Tyrosine kinase expressed in hepatocellular carcinoma (TEC), Interleukin-2 inducible T-cell kinase (ITK, also known as Tsk and Emt), Resting lymphocyte kinase (RLK, also known as TXK for Tyrosine Protein Kinase), Bruton's tyrosine kinase (BTK), Bone marrow kinase on the X-chromosome (BMX, also known as Etk) (Bradshaw JM Cell Signal; 2010, 22(8):1175-84). These intracellular kinases play important roles in the development and function of lymphocytes and myeloid cells (Horwood et al. Int. Rev. Immunol. 2012, 31(2):87-103, Felices Met al. Adv. Immunol; 2007, 93:145-84).

Tec kinases have unique N-termini followed by Src homology 3 (SH3) and Src homology 2 (SH2) protein interaction domains and a tyrosine kinase catalytic domain. Unique among the TEC kinases, TXK lacks a pleckstrin homology (PH) domain and has a nuclear localization signal sequence, which is responsible for nuclear translocation following TCR-mediated signaling (Mihara S., and Suzuki, N. Int. Rev. Imm; 2007, 26:333-348).

TXK has overlapping and unique activities compared with other members of the TEC kinase family. Like ITK, TXK is expressed in T-cells (Hu Q et al. J. Biol Chem. 1995, 270:1928-1934) and acts downstream of the T-cell receptor, however, TXK is preferentially expressed in Th1 cells in contrast to ITK which is expressed in Th2 polarized T cells (Sahu Net al. J. Immunol., 2008, 181:6125-6131). TXK specifically binds to the IFN-gamma promoter in human T cells to exert a positive effect on Th1 IFN-gamma gene transcription (Takeba Y., et al., J. Immunol., 2002, 168: 2365-2370). Furthermore, TXK phosphorylates a residue in the intracellular domain of CTLA4 which regulates PI3K activity (Schneider H. et al. Biochem Biophys Res Comm: 1998, 252(1), 14-19) suggesting that TXK may play a role in Treg function (Stumpf M et al. Eur J Immunol; 2014, 44(6): 1737-1746).

Increased expression of TXK has been reported in patients with Behcet's disease, an inflammatory disorder associated with increased inflammation and Th1 cytokine production (Suzuki Net al. 2006 Clin. Med. Res.4:147-151). Additionally, TXK positive lymphocytes have been observed in the inflamed synovium of rheumatoid arthritis patients (Mihara S., and Suzuki, N. Int. Rev. Imm. 2007; 26:333-348, 2007).

In addition, inhibition of TXK may be useful in combination with inhibition of other kinases. For example, knockout of both ITK and TXK produces stronger effects on T-cell function than knockout of either kinase alone (Schaeffer et al. 1999 Science 284:638-641; Felices et al. 2008 J. Immunol. 180:3007-3018). Furthermore, while ITK −/− mice have impaired in NKT cell generation this defect is exacerbated in the absence of both TXK and ITK (Felices M. et al. 2008, J Immunol. 180:3007-3018).

Specific inhibitors of TXK may be useful in treatment of diseases involving dysregulation of T-cells with increased safety and tolerability due to reduced off-target effects. These diseases may include hypersensitivity reactions, autoimmune disease, inflammation and cancer.

### BRIEF SUMMARY

The present invention relates to a novel family of covalent kinases inhibitors. Compounds of this class have been found to have inhibitory activity against members of the TEC kinase family, particularly TXK. One aspect of the present invention is directed to a compound of Formula I:

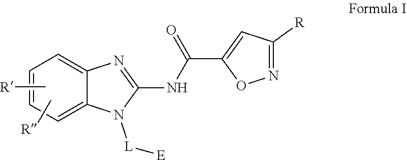

or a pharmaceutically acceptable salt, solvate, solvate of salt, isotope, prodrug, complex, or biologically active metabolite thereof; wherein

R is substituted or unsubstituted alkyl, substituted or unsubstituted alkenyl, substituted or unsubstituted alkynyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl;

L is:

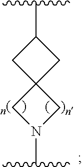

wherein
n is an integer from 1 to 3;
n′ is an integer from 1 to 3;

E is:

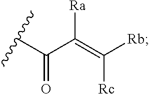

wherein
Ra, Rb, and Rc are each, independently, hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a 3- to 8-membered substituted or unsubstituted cycloalkyl ring or form a 3- to 8-membered substituted or unsubstituted heterocyclic ring, and Rc is hydrogen, halogen, —CN, substituted or unsubstituted alkyl, substituted or unsubstituted heteroalkyl, substituted or unsubstituted cycloalkyl, or substituted or unsubstituted heterocyclyl; or
Rb and Rc, taken together with the carbon atom to which they are attached, form a 3- to 8-membered substituted or unsubstituted cycloalkyl ring or form a 3- to 8-membered heterocyclic ring, and Ra is hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a triple bond, and Rc is hydrogen, halogen, —CN, substituted or unsubstituted alkyl, substituted or unsubstituted heteroalkyl, substituted or unsubstituted cycloalkyl, or substituted or unsubstituted heterocyclyl; and

R′ and R″ are each, independently, —X—Y; wherein
X is alkylene, -(alkylene)—NR¹—, -(alkylene)—NR²—, -(alkylene)—O—, —O—, —S—, —S(O)ₘ—, —NR¹—, —NR²—, —C(O)—, —C(O)O—, —C(O)NR¹—, —C(O)ONR¹—, or —S(O)ₘNR¹—;
   R¹ is selected from hydrogen, lower alkyl or lower cycloalkyl;
   R² is selected from —C(O)R³, —C(O)R³ or —S(O)ₘR³;
   R³ is selected from lower alkyl or lower cycloalkyl;
   m is an integer from 1 to 2; or
X is a bond; and
Y is hydrogen, halogen, substituted or unsubstituted alkyl, substituted or unsubstituted alkenyl, substituted or unsubstituted alkynyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl; or

R′ and R″ taken together with the carbon atoms to which they are attached form a 3- to 8-membered substituted or unsubstituted cycloalkyl ring, or a 3- to 8-membered substituted or unsubstituted heterocyclyl ring.

In another aspect provided herein a pharmaceutical composition comprising a compound disclosed herein, and/or a pharmaceutically acceptable salt, solvate, solvate of salt, stereoisomer, tautomer, isotope, prodrug, complex or biologically active metabolite thereof; and at least one pharmaceutically acceptable carrier or excipient.

In another aspect the present invention provides a pharmaceutical combination comprising a compound of Formula I disclosed herein or a pharmaceutically acceptable salt, solvate, solvate of salt, stereoisomer, tautomer, isotope, prodrug, complex or biologically active metabolite thereof and at least one additional active pharmaceutical ingredient for the treatment or prevention of cancer, autoimmune diseases, allergic diseases, inflammatory diseases or viral infection in combination therapy.

Another aspect of the present invention provides a pharmaceutical composition comprising the compound disclosed herein, or a pharmaceutically acceptable salt solvate, solvate of a salt, stereoisomer, tautomer, isotope, prodrug, complex or biologically active metabolite thereof, for use in treatment with at least one additional active pharmaceutical ingredient for the treatment of cancer, autoimmune diseases, allergic diseases, inflammatory diseases or viral infection wherein: said additional active pharmaceutical ingredient is appropriate for the disease being treated; and said additional active pharmaceutical ingredient is administered together with said composition as a single dosage form or separately from said composition as part of a multiple dosage form.

In another aspect the present invention provides a pharmaceutical combination comprising a compound of Formula I disclosed herein or a pharmaceutically acceptable salt, solvate, solvate of salt, stereoisomer, tautomer, isotope, prodrug, complex or biologically active metabolite thereof and at least one additional active pharmaceutical ingredient for the treatment or prevention of cancer, autoimmune diseases, allergic diseases, inflammatory diseases or viral infection in combination therapy.

Another aspect of the present invention provides the synthetic methods used to prepare compounds of Formula I of the present invention and are not intended to be limiting.

Another aspect of the present invention provides a method for treating a subject suffering from a protein kinase mediated disease or condition, comprising administering to the subject a therapeutically effective amount of the compound disclosed herein, or a pharmaceutically acceptable salt solvate, solvate of a salt, stereoisomer, tautomer, isotope, prodrug, complex or biologically active metabolite thereof in combination with at least one pharmaceutically acceptable carrier.

In yet another aspect, provided herein are methods of preventing or treating a disease treatable by inhibition of TXK (RLK) in a patient which comprises administering to the patient a pharmaceutical composition consisting of a compound disclosed herein and or a pharmaceutically acceptable salt thereof in a therapeutically effective amount and one or more pharmaceutically acceptable excipients. In one embodiment of this aspect the patient suffers from a disease or disorder that can be treated by kinase inhibition.

Another aspect of the present invention provides a method of modulating kinase activity in a subject comprising administering a therapeutically effective amount of the compound disclosed herein, or a pharmaceutically acceptable salt solvate, solvates of a salt, stereoisomer, tautomer, isotope, prodrug, complex or biologically active metabolite thereof.

Another aspect of the present invention provides a method of inhibiting protein kinase in a cell or tissue comprising contacting the cell or tissue with the compound disclosed herein, or a pharmaceutically acceptable salt solvate, solvates of a salt, stereoisomer, tautomer, isotope, prodrug, complex or biologically active metabolite thereof.

Another aspect of the present invention provides a method of inhibiting protein kinase activity in a subject, comprising administering a therapeutically effective amount of the compound disclosed herein, or a pharmaceutically acceptable salt solvate, solvates of a salt, stereoisomer, tautomer, isotope, prodrug, complex or biologically active.

Another aspect of the present invention provides a method of treating a subject suffering from cancer, autoimmune diseases, allergic diseases, inflammatory diseases viral infection or combinations thereof, wherein the enzymatic activity of TXK (RLK) is reduced by administering to the subject a therapeutically effective amount of the compound disclosed herein or a pharmaceutically acceptable salt solvate, solvate of a salt, stereoisomer, tautomer, isotope, prodrug, complex or biologically active metabolite thereof.

In one embodiment the present invention provides a method of treatment wherein further comprising administering of a therapeutically effective amount of at least one additional active pharmaceutical ingredient for the treatment of cancer, autoimmune diseases, allergic diseases, inflammatory diseases, neurological disorders or viral infection in combination therapy. The additional active pharmaceutical ingredient is administered together with the compounds of Formula I or a pharmaceutically acceptable salt, solvate, solvate of salt, stereoisomer, tautomer, isotope, prodrug, complex or biologically active metabolite thereof, as a single dosage form or separately, as part of a multiple dosage form. The additional active pharmaceutical ingredient is selected from the group comprising: steroids, leukotriene antagonists, anti-histamines, anti-cancer, anti-viral, anti-biotic agents, protein kinase inhibitors or combinations thereof.

Another aspect of the present invention provides a probe comprising the compound as disclosed therein which is covalently conjugated to a detectable label or affinity tag for said compound. The probe, wherein the detectable label is selected from the group consisting of a fluorescent moiety, a chemiluminescent moiety, a paramagnetic contrast agent, a metal chelate, a radioactive isotope containing moiety and biotin.

The administration of a compound of the present invention may be by any appropriate means known in the field, including systemic and localized administration. Prior to administration, the compounds may be formulated as compositions suitable for pharmaceutical or clinical use. Such compositions may comprise appropriate carriers or excipients, such as those for topical, inhalation, or systemic administration. The compound of the present invention may be administered alone or in combination with one or more pharmaceutically acceptable active for the treatment or prevention of a protein kinase mediated condition.

All publications, patent applications, patents and other references mentioned herein are incorporated by references in their entirety.

Other features, objects, and advantages of the invention(s) disclosed herein will be apparent from the description and drawings, and from the claims.

### DETAILED DESCRIPTION

The present invention relates to a novel covalent kinase inhibitor of Formula I:

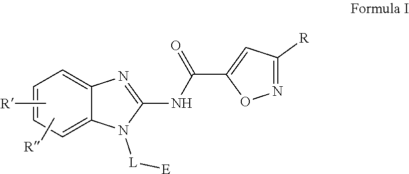

or a pharmaceutically acceptable salt, solvate, solvate of salt, isotope, prodrug, complex, or biologically active metabolite thereof; wherein

R is substituted or unsubstituted alkyl, substituted or unsubstituted alkenyl, substituted or unsubstituted alkynyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl;

L is:

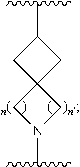

wherein
n is an integer from 1 to 3;
n′ is an integer from 1 to 3;

E is:

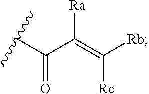

wherein
Ra, Rb, and Rc are each, independently, hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a 3- to 8-membered substituted or unsubstituted cycloalkyl ring or form a 3- to 8-membered substituted or unsubstituted heterocyclic ring, and Rc is hydrogen, halogen, —CN, substituted or unsubstituted alkyl, substituted or unsubstituted heteroalkyl, substituted or unsubstituted cycloalkyl, or substituted or unsubstituted heterocyclyl; or
Rb and Rc, taken together with the carbon atom to which they are attached, form a 3- to 8-membered substituted or unsubstituted cycloalkyl ring or form a 3- to 8-membered heterocyclic ring, and Ra is hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a triple bond, and Rc is hydrogen, halogen, —CN, substituted or unsubstituted alkyl, substituted or unsubstituted heteroalkyl, substituted or unsubstituted cycloalkyl, or substituted or unsubstituted heterocyclyl; and

R′ and R″ are each, independently, —X—Y; wherein
X is alkylene, -(alkylene)—NR¹—, -(alkylene)—NR²—, -(alkylene)—O—, —O—, —S—, —S(O)ₘ—, —NR¹—, —NR²—, —C(O)—, —C(O)O—, —C(O)NR¹—, —C(O)ONR¹—, or —S(O)ₘNR¹—;
   R¹ is selected from hydrogen, lower alkyl or lower cycloalkyl;
   R² is selected from —C(O)R³, —C(O)OR³ or —S(O)ₘR³;
   R³ is selected from lower alkyl or lower cycloalkyl;
   m is an integer from 1 to 2; or
X is a bond; and
Y is hydrogen, halogen, substituted or unsubstituted alkyl, substituted or unsubstituted alkenyl, substituted or unsubstituted alkynyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl; or

R′ and R″ taken together with the carbon atoms to which they are attached form a 3- to 8-membered substituted or unsubstituted cycloalkyl ring, or a 3- to 8-membered substituted or unsubstituted heterocyclyl ring.

An embodiment includes compounds of Formula I, where Ra, Rb and Rc are independently selected from the group consisting of hydrogen, -CN, halogen, and C₁ to C₃ substituted or unsubstituted alkyls.

An embodiment includes compounds of Forumla 1, where E is

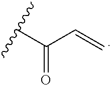 [SMILES: *C(=O)C=C]

An embodiment includes compounds of Formula I, where L-E is selected from

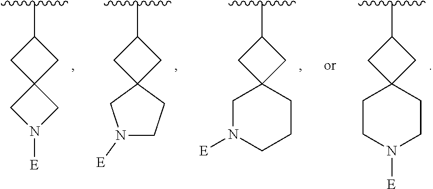

An embodiment includes compounds of Formula I, where L-E is

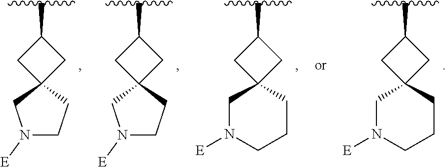

An embodiment includes compounds of Formula I, where L-E is

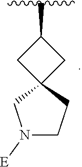

An embodiment includes compounds of Formula I, where L-E is

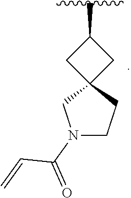 [SMILES: *[C@H]1C[C@@]2(CCN(C(=O)C=C)C2)C1]

An embodiment includes compounds of Formula I, where R′ is selected from —CH₂—Y wherein Y is as defined above and R″ is hydrogen.

One aspect of the present invention is directed to a compound of Formula II:

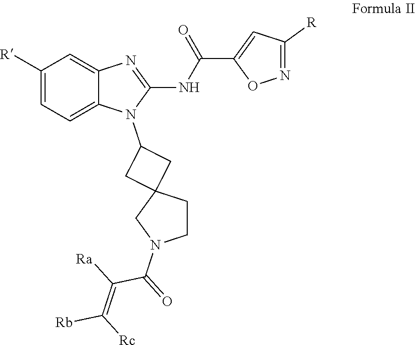

or a pharmaceutically acceptable salt, solvate, solvate of salt, isotope, prodrug, complex, or biologically active metabolite thereof; wherein

R is substituted or unsubstituted alkyl, substituted or unsubstituted cycloalkyl, substituted or unsubstituted heterocyclyl, substituted or unsubstituted aryl, or substituted or unsubstituted heteroaryl;

Ra, Rb, and Rc are each, independently, hydrogen, halogen, —CN, substituted or unsubstituted alkyl, substituted or unsubstituted heteroalkyl, substituted or unsubstituted cycloalkyl, or substituted or unsubstituted heterocyclyl; and

R′ is —X—Y; wherein
X is a bond or alkylene; and
Y is hydrogen, substituted or unsubstituted cycloalkyl, substituted or unsubstituted heterocyclyl, substituted or unsubstituted aryl, or substituted or unsubstituted heteroaryl.

An embodiment includes compounds of Formula II, where Ra, Rb, and Rc are each hydrogen.

An embodiment includes compounds of Formula II, where R′ is selected from —CH₂—Y wherein Y is as defined above. In a more preferred embodiment, Y is substituted or unsubstituted heterocyclyl. In one embodiment, Y is alkyl substituted heterocyclyl.

An embodiment includes compounds of Formula II, where R is unsubstituted alkyl. In a more preferred embodiment, R is methyl, ethyl, or propyl.

An embodiment includes compounds of Formula II, where R is unsubstituted cycloalkyl. In a more preferred embodiment, R is cyclobutyl, cyclopentyl, or cyclohexyl.

An embodiment includes compounds of Formula II, where R is unsubstituted aryl. In a more preferred embodiment, R is phenyl.

Compounds of Formula I and II can exist as tautomers. For example, compounds of Formula I can exist in the following tautomeric form:

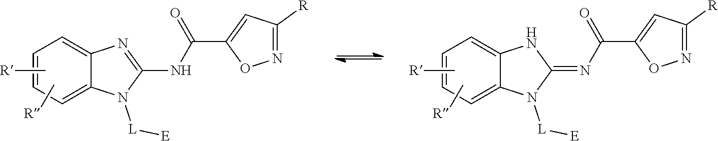

wherein R, R′, R″, L and E are as defined herein.

The compounds of the present invention may have activity as inhibitors of protein kinases including tyrosine protein kinases. Most particularly, compounds of the present invention may inhibit TXK (RLK) enzyme and TXK (RLK)-dependent cellular functions.

In an embodiment of the present invention compounds of Formula I may be formulated into a pharmaceutical composition which comprises an effective amount of a compound of the present invention with a pharmaceutically acceptable diluent or carrier.

According to the present invention there is provided a pharmaceutical composition which comprises a compound of Formula I, or a pharmaceutically acceptable salt or solvate thereof, in association with at least one pharmaceutically acceptable excipient, diluent or carrier.

The pharmaceutical compositions may be in a conventional pharmaceutical form suitable for oral administration (e.g., tablets, capsules, granules, powders and syrups), parenteral administration (e.g., injections (intravenous, intramuscular, or subcutaneous)), drop infusion preparations, inhalation, eye lotion, topical administration (e.g., ointment), or suppositories. Regardless of the route of administration selected, the compounds may be formulated into pharmaceutically acceptable dosage forms by conventional methods known to those skilled in the art.

The term “compound” refers also to its pharmaceutically acceptable salt, solvate, solvate of salt, stereoisomer, tautomer, isotope, prodrug, complex or biologically active metabolite thereof.

The term “pharmaceutically effective amount” refers to any amount of the composition for the prevention and treatment of humans that is effective in preventing or treating a disease or condition associated with protein kinase activity.

The term “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein means a pharmaceutically acceptable material, composition, or vehicle, such as a liquid or solid filler, diluent, excipient, solvent or encapsulating material. Each carrier must be acceptable in the sense of being compatible with the other ingredients of the formulation, including the active ingredient, and not injurious or harmful to the patient. Some examples of materials which may serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. For oral formulations, “pharmaceutically acceptable carrier” such as cellulose, calcium silicate, corn starch, lactose, sucrose, dextrose, calcium phosphate, stearic acid, magnesium stearate, calcium stearate, gelatin, talc, surfactants, suspending agents, emulsifiers, diluents, and others may be used. For injectable formulations, “pharmaceutically acceptable carrier” such as water, saline, glucose solution, glucose solution analogs, alcohols, glycols, ethers (e.g., polyethylene glycol 400), oils, fatty acids, fatty acid esters, glycerides, surfactants, suspending agents, emulsifiers, and others may be used.

The term “pharmaceutically acceptable salt” refers to the relatively non-toxic, inorganic and organic acid addition salts of the compound(s). These salts may be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like (See, for example, Berge et al., “Pharmaceutical Salts”, J. Pharm. Sci. 1977, 66: 1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts may likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “spirocycle”, as used herein, refers to bicyclic rings system connected through just one atom. The rings can be different or identical. The connecting atom, also called spiroatom, is preferably a quaternary carbon. Spirocycle may be optionally substituted with one or more substituents as defined herein.

The term “alkyl”, as used herein, refers to a saturated hydrocarbon chain. Alkyl chains may be straight or branched. Alkyl chains may be optionally substituted with one or more substituents as defined herein. Representative alkyl groups include methyl, ethyl, propyl, (n-propyl and isopropyl) butyl (n-butyl, t-butyl and isobutyl), pentyl (n-pentyl and isopentyl), hexyl and the like. In certain preferred embodiments, alkyl substituents are lower alkyl groups, e.g., having between 1 to 6 carbon atoms to 1 to 3 carbon atoms.

The term “alkenyl”, as used herein, refers to an unsaturated hydrocarbon chain analogous in length and possible substitution to the “alkyl” described above, but that contain at least one double bond. Representative alkenyl groups include vinyl, propen-2-yl, crotyl, isopenten-2-yl, 1,3-butadien-2-yl, 2,4-pentadienyl, and 1,4-pentadien-3-yl. In certain preferred embodiments, alkenyl substituents are lower alkenyl groups, e.g., having from 2 to 6 carbon atoms.

The term “alkynyl”, as used herein, refers to an unsaturated hydrocarbon chain analogous in length and possible substitution to the “alkyl” described above, but that contain at least one triple bond. Representative alkynyl groups include ethynyl, 1- and 3-propynyl, and 3-butynyl. In certain preferred embodiments, alkynyl substituents are lower alkyl groups, e.g., having from 2 to 6 carbon atoms.

The term, “alkylene”, as used herein, refers to an alkyl group with two open valencies.

The term “heteroalkyl”, as used herein, refers to a saturated or partially saturated chain containing one to four heteroatoms selected from the group consisting of O, N and S, and wherein the nitrogen and sulfur atoms may optionally be oxidized and the nitrogen atom may optionally be quaternized. Heteroalkyl chains may be straight or branched. Heteroalkyl chains may be optionally substituted with one or more substituents as defined herein. The heteroatom(s) O, N and S may be placed at any interior position of the heteroalkyl group. Up to two heteroatoms may be consecutive.

The term “cycloalkyl”, as used herein, alternatively “carbocycle” and “carbocyclyl” refers to a saturated or partially saturated non-aromatic ring, more preferably 3- to 8-membered ring, in which each atom of the ring is carbon or; refers to a spirocycle where each ring is a saturated or partially saturated hydrocarbon ring and the spiro atom is carbon. The term “cycloalkyl”, “carbocycle” or “carbocyclyl” also include polycyclic ring systems having two or more cyclic rings in which two or more carbons are common to two adjoining rings wherein at least one of the rings is cycloalkyl, e.g., the other cyclic rings can be aryls, heteroaryls, and/or heterocyclyls. Representative cycloalkyl rings include cyclopropyl, cyclobutyl, cyclopentyl, cyclohexyl, 1-cyclohexenyl, 3-cyclohexen-1-yl, cycloheptyl, tetrahydronaphthyl, indanyl, adamantly and combinations thereof. Cycloalkyl rings may be optionally substituted with one or more substituents as defined herein. In certain preferred embodiments, cycloalkyl substituents are lower cycloalkyl, e.g., refer to 3- to 8-membered ring, in which each atom of the ring is carbon or; refers to a spirocycle where each ring is a saturated or partially saturated hydrocarbon ring and the spiro atom is carbon.

The term “heterocyclyl” alternatively “heterocyclic”, as used herein, refers to non-aromatic ring structures, more preferably 3- to 8-membered rings, whose ring structures include one to four heteroatoms or; refers to a spirocycle where the bicyclic rings system contains 1 to 4 heteroatoms. Heterocyclyl rings may be optionally substituted with one or more substituents as defined herein. The term “heterocyclyl” or “heterocyclic” also include polycyclic ring systems having two or more cyclic rings in which two or more carbons are common to two adjoining rings wherein at least one of the rings is heterocyclic, e.g., the other cyclic rings can be cycloalkyls, aryls and/or heteroaryls. Heterocyclyl groups include, for example, tetrahydrofuran, piperidine, piperazine, pyrrolidine, morpholine, lactones, lactams and combinations thereof.

The term “aryl”, as used herein, refers to 5-, 6-, and 7-membered aromatic rings in which each atom of the ring is carbon. Aryl rings may be optionally substituted with one or more substituents as defined herein. The term “aryl” also includes polycyclic ring systems having two or more cyclic rings in which two or more carbons are common to two adjoining rings wherein at least one of the rings is aryl, e.g., the other cyclic rings can be cycloalkyls, heteroaryls, and/or heterocyclyls. Aryl groups include, for example, benzene, naphthalene, phenanthrene, anthracene and combinations thereof.

The term “heteroaryl” or Het, as used herein, refers to 5-, 6-, and 7-membered aromatic rings whose ring structures include one to four heteroatoms. Heteroaryl rings may be optionally substituted with one or more substituents as defined herein. The term “heteroaryl” also includes polycyclic ring systems having two or more cyclic rings in which two or more carbons are common to two adjoining rings wherein at least one of the rings is heteroaryl, e.g., the other cyclic rings can be cycloalkyls, aryls and/or heterocyclyls. Heteroaryl groups include, for example, pyrrole, furan, thiophene, imidazole, isoxazole, oxazole, thiazole, triazole, pyrazole, pyridine, pyrazine, pyridazine and pyrimidine, and combinations thereof.

The terms “polycyclyl” alternatively “polycyclic”, as used herein, refer to two or more rings (e.g., cycloalkyls, aryls, heteroaryls, and/or heterocyclyls) in which two or more carbons are common to two adjoining rings, e.g., the rings are “fused rings”. Polycyclyl rings may be optionally substituted with one or more substituents as defined herein.

The term “aralkyl”, as used herein, refers to an alkyl group substituted with an aryl group, for example —(CH₂)ₚ—Ar and p is an integer from 1 to 8.

The term “heteroaralkyl”, as used herein, refers to an alkyl group substituted with a heteroaryl group, for example —(CH₂)ₚ-Het and p is an integer from 1 to 8.

The term “alkoxy”, as used herein, refers to an alkyl ether substituent, wherein the term alkyl is as defined therein. Representative alkoxy groups include methoxy, ethoxy, propoxy, tert-butoxy and combinations thereof.

The term “ether”, as used herein, refers to an oxy group bridging two moieties linked at carbon atoms.

The term “alkoxyalkyl”, as used herein, refers to an alkyl group substituted with an alkoxy group, thereby forming ether.

The term “halo” or “halogen”, as used herein, refers to fluorine, chlorine, bromine and iodine.

The term “heteroatom”, as used herein, refers to an atom of any element other than carbon or hydrogen. Preferred heteroatoms are nitrogen, oxygen, and sulfur.

The term “hydrocarbon”, as used herein, refers to a group consisting entirely of carbon and hydrogen.

The term, “haloalkyl”, as used herein, refers to an alkyl substituent wherein one or more hydrogens are replaced by a halogen.

The term “carbonyl”, as used herein, when alone includes formyl —CH(O) and in combination is a —C(O) group.

The term “carboxyl”, alternatively “carboxy”, as used herein, refers to —C(O)OH or the corresponding “carboxylate” anion, such as in a carboxylic acid salt.

The term “acyl”, as used herein, refers to —C(O)R wherein R is alkyl, heteroalkyl, haloalkyl, cycloalkyl, heterocyclyl, aryl or heteroaryl as defined therein. Representative acyl groups include acetyl, trifluoroacethyl, benzoyl, and combinations thereof.

The term “alkoxycarbonyl”, as used herein, refers to —C(O)OR wherein R is alkyl as defined therein. Representative alkoxycarbonyl groups include methoxycarbonyl, ethoxycarbonyl, and combinations thereof.

The term “alkylthio”, as used herein, refers to a thioether —SR wherein R is alkyl as defined therein. Representative alkylthio groups include methylthio, ethylthio and combinations thereof.

The term “sulfonate”, as used herein, refers to a salt or ester of a sulfonic acid —OSO₂R wherein R is alkyl, heteroalkyl, haloalkyl, cycloalkyl, heterocyclyl, aryl or heteroaryl as defined therein. Representative sulfonate groups include mesylate, besylate, tosylate, and combinations thereof.

The term “sulfonyl”, as used herein, refers to —SO₂R wherein R is alkyl, heteroalkyl, haloalkyl, cycloalkyl, heterocyclyl, aryl or heteroaryl as defined therein. Representative sulfonate groups include methylsufonyl, ethylsulfonyl, and combinations thereof.

The term “sulfamoyl”, as used herein, refers to —SO₂NH₂.

The term “sulfonamido”, as used herein, refers to —S(O)₂NRR′ wherein R and R′ are independently selected from alkyl, heteroalkyl, haloalkyl, cycloalkyl, heterocyclyl, aryl and heteroaryl as defined above. R and R′ may combine to form a heterocyclyl ring.

The term “amino”, as used herein, refers to —NRR′ wherein R and R′ are independently selected from hydrogen, alkyl, heteroalkyl, haloalkyl, cycloalkyl, heterocyclyl, aryl and heteroaryl as defined therein. R and R′ may combine to form a heterocyclyl ring.

The term “amido” alternatively “amide”, as used herein, refers to —C(O)NRR′ wherein R and R′ are independently selected from hydrogen, alkyl, heteroalkyl, haloalkyl, cycloalkyl, heterocyclyl, aryl or heteroaryl as defined therein. R and R′ may combine to form an heterocyclyl ring.

The term “substituted” refers to moieties having substituents replacing hydrogen on one or more atoms of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms.

Substituents can include, for example, an alkyl, an alkenyl, an alkynyl, a haloalkyl, a heteroalkyl, a cycloalkyl, a heterocyclyl, an aryl, a heteroaryl, a halogen, a hydroxyl, a carbonyl , carboxyl, an alkoxycarbonyl, a formyl, or an acyl, a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl. It will be understood by those skilled in the art that the substituents can themselves be substituted, if appropriate.

As used herein, the term “probe” means a compound of the invention which is labeled with either a deTECtable label or an affinity tag, and which is capable of binding, either covalently or non-covalently, to a protein kinase domain. When, for example, the probe is non-covalently bound, it may be displaced by a test compound. When, for example, the probe is bound covalently, it may be used to form cross-linked adducts, which may be quantified and inhibited by a test compound. As used herein, the term “affinity tag” means a ligand or group, linked either to a compound of the present invention or to a protein kinase domain, that allows the conjugate to be extracted from a solution.

The term “prodrug” denotes a compound that is a drug precursor which, upon administration to a subject, is converted within the body into a compound of Formula I. Prodrugs of compounds of Formula I or pharmaceutically acceptable salts or solvates thereof are within the scope of this disclosure.

The term “subject” or “patient” means a human or an animal subject for prevention or treatment.

In an embodiment the use is ex vivo, for example in vitro, such as an in vitro assay.

Compounds of the invention also include all isotopes of atoms present in the intermediates and/or final compounds. Isotopes include those atoms having the same atomic number but different mass numbers. For example, isotopes of hydrogen include deuterium and tritium.

### Therapeutic Uses and Applications

The compounds of the present invention may have potential utility as inhibitors of protein kinase activity and are suitable for use in therapy.

An aspect of the present invention provides a method of inhibiting protein kinase activity in a cell, the method consisting of administering to said cell compound of Formula I as defined herein, or a pharmaceutically acceptable salt, solvate, solvate of salt, stereoisomer, tautomer, isotope, prodrug, complex or biologically active metabolite thereof.

In a further aspect, the present invention provides a method of inhibiting protein kinase in vitro or in vivo, said method consisting of contacting a cell with an effective amount of a compound of Formula I, or a pharmaceutically acceptable salt or solvate thereof, as defined herein.

A further aspect of the present invention provides a method of inhibiting protein kinase activity in a human or animal subject for treatment or prevention of protein kinase mediated disease, the method consisting of administering to said subject an effective amount of a compound of Formula I as defined herein, or a pharmaceutically acceptable salt or solvate thereof.

The term “protein kinase mediated disease” is used herein associated with abnormal or undesirable cellular responses triggered or maintained by protein kinase-mediated events. Furthermore, aberrant activation, mutation or excessive expressions of various protein kinases are implicated in the mechanism of multiple diseases and disorders. These diseases include, but are not limited to cancer, autoimmune disease, inflammation, viral infection and/or neurological disease.

In one embodiment, the protein kinase inhibited by compounds of the present invention is RLK (TXK).

The compounds of the present invention may be suitable for use in the treatment of or prevention of diseases that involve TXK (RLK) i.e. diseases that involve T cells and/or NK cells, for example, cancer, autoimmune diseases, allergic diseases, inflammatory diseases, viral infection and combinations thereof.

In one embodiment, a compound disclosed herein and/or pharmaceutically acceptable salt thereof is administered to a patient in need or recognized need thereof to prevent or treat an inflammatory disorder. In another embodiment, a compound disclosed herein and/or pharmaceutically acceptable salt thereof is administered to a patient in need or recognized need thereof to prevent or treat an inflammatory disorder characterized by excessive or undesired cytokine activity or production. In yet another embodiment, a compound and/or pharmaceutically acceptable salt thereof is administered to a patient in need or recognized need thereof to prevent or treat lung inflammation, allergic asthma, pneumonia, psoriasis, atopic dermatitis or a combination thereof. In yet another embodiment a compound and/or pharmaceutically acceptable salt thereof is administered to a patient in need of or recognized need thereof to prevent or treat uveitis or dry eye disease.

Examples of an autoimmune disease in the present invention include arthritis, systemic lupus erythematosus, rheumatoid arthritis, psoriasis, psoriatic arthritis, Still's disease, juvenile arthritis, type I diabetes, inflammatory bowel disease, myasthenia gravis, Hashimoto's thyroiditis, Ord's thyroiditis, Basedow's disease, Sjogren's syndrome, multiple sclerosis, Guillain-Barre syndrome, acute disseminated encephalomyelitis, Addison disease, opsoclonus-myoclonus syndrome, ankylosing spondylitis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's disease, Takayasu arteritis, temporal arteritis, warm autoimmune hemolytic anemia, Wegener granuloma, alopecia universalis, Burchett disease, chronic fatigue syndrome, dysautonomia, endometriosis, interstitial cystitis, myotonia, vulvodynia, pemphigus, and combinations thereof.

Examples of an allergic disease in the present invention include allergy, anaphylaxis, allergic conjunctivitis, allergic rhinitis, atopic dermatitis and combinations thereof.

Examples of an inflammatory disease in the present invention include asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, epicondylitis, epididymitis, fasciitis, fibrositis, gastritis, gastroenteritis, hepatitis, hidradenitis suppurativa, inflammatory bowel disease, laryngitis, mastitis, meningitis, myelitis, myocarditis, myositis nephritis, oophoritis, orchitis, osteitis, osteoarthritis, pancreatitis, parotitis, pericarditis, peritonitis, pharyngitis, pleuritis, phlebitis, pneumonia, proctitis, prostatitis, pyelonephritis, rhinitis, salpingitis, sinusitis, stomatitis, synovitis, tendinitis, tonsillitis, uveitis, vaginitis, vasculitis, vulvitis, and combinations thereof.

Examples of an infection include HIV/AIDS, influenza and combinations thereof.

Examples of cancer in the present invention include T-cell lymphomas and T-cell leukemias including peripheral T-cell lymphoma, Sezary syndrome/cutaneous T-cell lymphoma, acute lymphoblastic leukemia, and adult T-cell leukemia/lymphoma. Additional examples include NK/T-cell lymphoma, nasal type and aggressive NK-cell leukemia as well as melanoma and hepaptocellular carcinoma.

In one embodiment, the compound of Formula I or pharmaceutically acceptable salt, solvate, solvate of salt, stereoisomer, tautomer, isotope, prodrug, complex, or biologically active metabolite thereof, is acting by inhibiting one or more of the host cell kinases involved in cell proliferation, cell survival, viral replication, autoimmunity, an inflammatory disease or an infectious disease.

In further aspect of the present invention, the compound of Formula I or pharmaceutically acceptable salt, solvate, solvate of salt, stereoisomer, tautomer, isotope, prodrug, complex, or biologically active metabolite thereof, is acting as inhibitor of cell kinases as anti-inflammatory, autoimmune modulators or anti-cancer agents.

In a further aspect of the present invention, the compound of Formula I or pharmaceutically acceptable salt, solvate, solvate of salt, stereoisomer, tautomer, isotope, prodrug, complex, or biologically active metabolite thereof, is acting by inhibiting one or more of the host cell kinases involved in T-cell function proliferation or polarization.

The compounds of Formula I or pharmaceutically acceptable salt, solvate, solvate of salt, stereoisomer, tautomer, isotope, prodrug, complex, or biologically active metabolite thereof and pharmaceutically acceptable compositions of the present invention can be employed in combination therapies, the compounds and pharmaceutically acceptable compositions may have potential utility in combination with other therapies for the treatment of cancer, viral infections, immune, inflammatory, neurological diseases, proliferative and/or allergic disorders. Example includes but not limited to co-administration with steroids, leukotriene antagonists, anti-histamines, anti-cancer, anti-viral, anti-biotic agents and/or other protein kinase inhibitors. The anti-cancer agent may be selected from the group consisting of: cell signal transduction inhibitors, mitosis inhibitors, alkylating agents, anti-metabolites, intercalating anticancer agents, topoisomerase inhibitors, immunotherapeutic agents, anti-hormonal agents, and/or a mixture thereof. The additional active pharmaceutical ingredient used in the combination is appropriate for the disease being treated and said additional active pharmaceutical ingredient is administered together with the compounds of Formula I as a single dosage form or separately as part of a multiple dosage form. The term “combination” includes the simultaneous, sequential or separate use of the components, ingredients and/or compounds.

Another aspect of the present invention provides compounds of the present invention can be administered by any means suitable for the condition to be treated, which may depend on the need for site-specific treatment or quantity of drug to be delivered. Topical administration is generally preferred for skin-related diseases, and systematic treatment preferred for cancerous or pre-cancerous conditions, although other modes of delivery are contemplated. For example, the compounds may be delivered orally, such as in the form of tablets, capsules, granules, powders, and/or liquid formulations including syrups; topically, such as in the form of solutions, suspensions, gels, cream and/or ointments; sublingually; bucally; parenterally, such as by subcutaneous, intravenous, intramuscular and/or intrasternal injection or infusion techniques (e.g., as sterile injectable aqueous or non-aqueous solutions or suspensions); nasally such as by inhalation spray; rectally such as in the form of suppositories; or liposomally. Dosage unit formulations containing non-toxic, pharmaceutically acceptable vehicles and/or diluents may be administered. The compounds may be administered in a form suitable for immediate release, extended release, delayed release and/or controlled release. Immediate release and/or extended release may be achieved with suitable pharmaceutical compositions or, particularly in the case of extended release, with devices such as subcutaneous implants or osmotic pumps. The compounds may be administered in a form suitable for targeted delivery in which the drug is only active in the target area of the body (for example, in cancerous tissues) and sustained release formulations in which the drug is released over a period of time in a controlled manner from a formulation.

The compounds of the present invention are indicated both in the therapeutic and/or prophylactic treatment of the above-mentioned conditions. For the above-mentioned therapeutic and/or prophylactic uses the dosage administered will vary with the compound employed, the subject, the mode of administration, the treatment desired and/or the disorder indicated. The daily dosage may be between about 0.01 mg/kg to about 100 mg/kg and preferably from about 1 mg/kg to about 25 mg/kg, of the subject body weight per day, one or more times a day, to obtain the desired therapeutic effect.

The compounds of Formula I or pharmaceutically acceptable salt, solvate, solvate of salt, stereoisomer, tautomer, isotope, prodrug, complex, and/or biologically active metabolite thereof may be suitable for use in the preparation of a medicament for inhibiting a protein kinase activity selected from TXK (RLK) in a subject.

A pharmaceutical acceptable composition of the present invention may be obtained by conventional procedures using conventional pharmaceutical excipients, well known in the art. It may typically comprise pharmaceutically acceptable additives, carriers or excipients. The pharmaceutical composition of the present invention may be formulated in accordance with conventional methods, and may be prepared in the form of oral formulations such as tablets, pills, powders, capsules, syrups, emulsions, microemulsions and others, and/or parenteral formulations such as intramuscular, intravenous or subcutaneous administrations.

For oral formulations, carriers or additives such as cellulose, calcium silicate, corn starch, lactose, sucrose, dextrose, calcium phosphate, stearic acid, magnesium stearate, calcium stearate, gelatin, talc, surfactants, suspending agents, emulsifiers, diluents, and others may be used. Solid dosage forms for oral administration include capsules, tablets, pills, powders, and/or granules. Liquid dosage forms for oral administration include, but are not limited to, pharmaceutically acceptable emulsions, microemulsions, solutions, suspensions, syrups and elixirs. The liquid dosage forms may contain inert diluents and can also include adjuvants such as wetting agents, emulsifying and suspending agents, sweetening, flavoring, and perfuming agents.

For Injectable formulations, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Another aspect of the present invention provides a pharmaceutical composition comprising a compound of Formula I or a pharmaceutically acceptable salt, solvate, solvate of salt, stereoisomer, tautomer, isotope, prodrug, complex and/or biologically active metabolite thereof, and at least one pharmaceutically acceptable carrier, diluent or excipient.

The pharmaceutical composition of the present invention is for use in prevention or treatment of cancer, autoimmune diseases, allergic diseases, inflammatory diseases, graft-versus-host disease, thromboembolic diseases, neurological disorders, viral infections, bone-related diseases and/or combinations thereof.

In an embodiment of the present invention a compound of Formula I or a pharmaceutically acceptable salt, solvate, solvate of salt, stereoisomer, tautomer, isotope, prodrug, complex or biologically active metabolite thereof suitable for use in therapy, wherein a subject is suffering of a disease, disorder or condition in which one or more protein kinase family member activity is implicated. In an embodiment, the protein kinase is selected from RLK (TXK).

In an embodiment of the present invention a compound of Formula I or a pharmaceutically acceptable salt, solvate, solvate of salt, stereoisomer, tautomer, isotope, prodrug, complex and/or biologically active metabolite thereof, is for use in the treatment or prevention of cancer, autoimmune diseases, allergic diseases, inflammatory diseases, neurological disorders, and/or viral infection in combination therapy.

In an embodiment of the present invention a compound of Formula 1 or a pharmaceutically acceptable salt, solvate, solvate of salt, stereoisomer, tautomer, isotope, prodrug, complex or biologically active metabolite thereof, is for use in therapy, further comprising at least one additional active pharmaceutical ingredient for the treatment and/or prevention of cancer, autoimmune diseases, allergic diseases, inflammatory diseases, neurological disorders and/or viral infection in combination therapy. The additional active pharmaceutical ingredient is selected from the group consisting of : steroids, leukotriene antagonists, anti-histamines, anti-cancer, anti-viral, anti-biotic agents, protein kinase inhibitors, immune modulators, checkpoint inhibitors and a combination thereof, and wherein additional active pharmaceutical ingredient is administered together with the compounds of Formula I or a pharmaceutically acceptable salt, solvate, solvate of salt, stereoisomer, tautomer, isotope, prodrug, complex or biologically active metabolite thereof, as a single dosage form, or separately as part of a multiple dosage form.

In another aspect, the present invention relates to a compound of the invention as defined herein, or a pharmaceutically acceptable salt or solvate thereof, or a pharmaceutical composition as defined herein, for use in therapy or prevention of protein kinase mediated disease.

Compounds of the present invention, in any aspect or embodiment may be used in the treatment or prevention of cancer, autoimmune diseases selected from: rheumatoid arthritis, juvenile rheumatoid arthritis, osteoarthritis, ankylosing spondylitis, psoriatic arthritis, psoriasis vulgaris, pemphigus vulgaris, bullous pemphigoid, Sjogren's syndrome, systemic lupus erythematosus, discoid SLE, lupus nephritis, antiphospholipidosis, whipple, dermatomyositis, polymyositis, autoimmune thrombocytopenia, idiopathic thrombocytopenia purpura, thrombotic thrombocytopenia purpura, autoimmune (cold) agglutinin disease, autoimmune hemolytic anemia, cryoglobulinemia, autoimmune vasculitis, ANCA-associated vasculitis, scleroderma, systemic sclerosis, multiple sclerosis, chronic focal encephalitis, Guillian-Barre syndrome, chronic fatigue syndrome, mononucleosis, neuromyelitis optica, autoimmune uveitis, Grave' s disease, thyroid associated opthalmopathy, granulomatosis with microscopic polyangitis, Wegeners granulomatosis, idiopathic pulmonary fibrosis, sarcoidosis, idiopathic membranous nephropathy, IgA nephropathy, glomerulosclerosis , pancreatitis , type I diabetes or type II diabetes, allergic diseases, inflammatory diseases, neurological disorders and/or viral infection in combination therapy.

In another aspect, the present invention relates to a compound of the invention as defined herein, or a pharmaceutically acceptable salt or solvate thereof, or a pharmaceutical composition as defined herein, for use in the treatment of subjects suffering from a protein kinase mediated diseases or conditions. In an embodiment, the protein kinase is selected from TXK (RLK).

Another aspect of the present invention provides a use of the compound of Formula I or a pharmaceutically acceptable salt or solvate thereof as an inhibitor of protein kinase an embodiment, the protein kinase is selected from TXK (RLK). In an embodiment the use is ex vivo, for example in vitro, such as an in vitro assay.

In another aspect, the present invention relates to the use of a compound of the invention as defined herein, or a pharmaceutically acceptable salt or solvate thereof, in the manufacture of a medicament for use in subjects for the treatment or prevention of protein kinase mediated diseases or conditions, for the treatment of cancer, autoimmune diseases, allergic diseases, inflammatory diseases, graft-versus-host disease, thromboembolic diseases, neurological disorders, viral infections, bone-related diseases or combinations thereof. In an embodiment, the protein kinase is selected from I RLK (TXK).

In another aspect, the present invention relates to a method of treating or prevention of a disease or condition associated with protein kinase activity, said method comprising administering to a subject a therapeutically effective amount of a compound of the invention as defined herein, or a pharmaceutically acceptable salt or solvate thereof, or a pharmaceutical composition as defined herein. In an embodiment, the protein kinase is selected from RLK (TXK).

Another aspect of the present invention provides a compound, or a pharmaceutically acceptable salt or solvate thereof, or a pharmaceutical composition as defined herein, for use in the treatment of or prevention of diseases that involve TXK (RLK) kinase, i.e. diseases that involve B cells, T-cells and/or mast cells, for example, cancer, autoimmune diseases, allergic diseases, inflammatory diseases, graft-versus-host disease, thromboembolic diseases, bone-related diseases and the like.

A further aspect of the present invention provides the use of a compound, or a pharmaceutically acceptable salt or solvate thereof, in the manufacture of a medicament for use in the treatment or prevention of diseases that involve TXK (RLK) kinase, i.e. diseases that involve B cells, T-cells and mast cells, for example, cancer, autoimmune diseases, allergic diseases, inflammatory diseases, graft-versus-host disease, thromboembolic diseases, bone-related diseases and the like.

In another aspect, the present invention provides a method of treating or preventing a disease or condition, said method comprising administering to a subject a therapeutically effective amount of a compound of Formula I or a pharmaceutically acceptable salt or solvate thereof, or a pharmaceutical composition as defined herein. In a particular embodiment, the disease or conditions include allergic diseases, autoimmune diseases, inflammatory diseases, thromboembolic diseases, bone-related diseases, cancer, graft-versus-host disease, and the like.

Another aspect of the present invention provides a method of modulating kinase function, the method comprising contacting a cell with a compound of the present invention in an amount sufficient to modulate the enzymatic activity of TXK (RLK) kinase, thereby modulating the kinase function. The method may be ex vivo, for example in vitro.

Another aspect of the present invention provides a method of inhibiting cell proliferation or survival in vitro or in vivo, said method comprising contacting a cell with an effective amount of a compound as defined herein, or a pharmaceutically acceptable salt or solvate thereof.

In one embodiment the present invention provides a method of producing a protein kinase inhibitory effect in a cell or tissue, said method comprising contacting the cell or tissue with an effective amount of a compound of Formula I or a pharmaceutically acceptable salt or solvate thereof.

In other embodiment, the present invention provides a method of producing a protein kinase inhibitory effect in vivo, said method comprising administering to a subject an effective amount of a compound of Formula 1, or a pharmaceutically acceptable salt or solvate thereof.

Another aspect of the present invention provides a method of modulating the target kinase function, comprising:
contacting a cell or a protein kinase with a compound of the present invention in an amount sufficient of a compound of Formula 1, or a pharmaceutically acceptable salt or solvate thereof to modulate the target kinase function, thereby;
modulating the target kinase activity and signaling.

In yet another aspect, provided herein are methods of treating a disease treatable by inhibition of protein kinase in a patient which comprises administering to the patient a pharmaceutical composition comprising a compound Formula 1 disclosed herein and or a pharmaceutically acceptable salt thereof in a therapeutically effective amount and one or more pharmaceutically acceptable excipients. In one embodiment of this aspect the patient suffers from a disease or disorder that can be treated by kinase inhibition. The compound disclosed herein of Formula I or pharmaceutically acceptable salt thereof can inhibit TXK (RLK).

In another aspect the present invention provides a pharmaceutical combination comprising a compound of the present invention and at least one additional active pharmaceutical ingredient for the treatment or prevention of cancer, autoimmune diseases, allergic diseases, inflammatory diseases or viral infection in combination therapy.

In one embodiment the present invention provides a method of treatment wherein further comprising administering of a therapeutically effective amount of at least one additional active pharmaceutical ingredient for the treatment of cancer, autoimmune diseases, allergic diseases, inflammatory diseases, neurological disorders or viral infection in combination therapy. The additional active pharmaceutical ingredient is administered together with the compounds of Formula I as a single dosage form or separately as part of a multiple dosage form. The additional active pharmaceutical ingredient is selected from the group comprising: steroids, leukotriene antagonists, anti-histamines, anti-cancer, anti-viral, anti-biotic agents, protein kinase inhibitors or combinations thereof.

The administration of a compound of the present invention may be by any appropriate means known in the field, including systemic and localized administration. Prior to administration, the compounds may be formulated as compositions suitable for pharmaceutical or clinical use. Such compositions may comprise appropriate carriers or excipients, such as those for topical, inhalation, or systemic administration. The compound of the present invention may be administered alone or in combination with one or more pharmaceutically acceptable active for the treatment or prevention of a protein kinase mediated condition.

The compounds object of the present invention may be administered to a mammal 1 to 4 times a day. A dosage may be between 0.01-100 mg/kg body weight/day of the compound object of the present invention may be administered to a patient receiving these compositions. The dose can vary within wide limits and is to be suited to the individual conditions in each individual case. For the above uses the appropriate dosage will vary depending on the mode of administration, the particular condition to be treated and the effect desired. Preferably a dose of 1 to 50 mg/kg body weight/day may be used.

In an embodiment of the present invention suitable dosage rates for a subject, for example humans, are of the order of from about 10 mg to 3 g/day, administered orally once, or divided doses, such as 2 to 4 times a day, or in sustained release form. For topical delivery, depending on the permeability of the skin, the type and the severity of the disease and dependent on the type of formulation and frequency of application, different concentrations of active compounds within the medicament can be sufficient to elicit a therapeutic effect by topical application. Preferably, the concentration of an active compound pharmaceutically acceptable salts, solvates, solvates of salts, stereoisomers, tautomers, isotopes, prodrugs, complexes or biologically active metabolites thereof, within a medicament according to the present invention is in the range of between 1 μmol/L and 100 mmol/L.

In further aspect of the present invention, the compound of Formula I or pharmaceutically acceptable salts, solvates, solvates of salts, stereoisomers, tautomers, isotopes, prodrugs, complexes, or biologically active metabolites thereof, act as inhibitors of cell kinases as anti-inflammatory, anti-cancer, anti-viral and as antithrombotic agents.

The compounds and/or pharmaceutically acceptable salts of the present invention may be administered in combination with one or more other drugs in the treatment of diseases or conditions for which compounds of the present disclosure or the other drugs may have utility, where the combination of the drugs together are safer or more effective than either drug alone. Such other drug(s) may be administered, by a route and in an amount commonly used therefore, contemporaneously or sequentially with a compound of the present disclosure. When a compound and/or pharmaceutically acceptable salt of the present disclosure is used contemporaneously with one or more other drugs, a pharmaceutical composition in unit dosage form containing such other drugs and the compound and/or pharmaceutically acceptable salt of the present disclosure is preferred. However, the combination therapy may also include therapies in which the compound and/or pharmaceutically acceptable salt of the present disclosure and one or more other drugs are administered on different overlapping schedules. It is also contemplated that when used in combination with one or more other active ingredients, the compounds and/or pharmaceutically acceptable salts of the present disclosure and the other active ingredients may be used in lower doses than when each is used singly. Accordingly, the pharmaceutical compositions of the present disclosure also include those that contain one or more other active ingredients, in addition to a compound and/or pharmaceutically acceptable salt of the present disclosure.

The above combinations include combinations of a compound of the present disclosure not only with one other active compound, but also with two or more other active compounds. Likewise, compounds and/or pharmaceutically acceptable salts of the present disclosure may be used in combination with other drugs that are used in the prevention, treatment, control, amelioration, or reduction of risk of the diseases or conditions for which compounds of the present disclosure are useful. Such other drugs may be administered, by a route and in an amount commonly used therefore by those skilled in the art, contemporaneously or sequentially with a compound and/or pharmaceutically acceptable salt of the present disclosure. When a compound and/or pharmaceutically acceptable salt of the present disclosure is used contemporaneously with one or more other drugs, a pharmaceutical composition containing such other drugs in addition to the compound and/or pharmaceutically acceptable salt of the present disclosure is preferred. Accordingly, the pharmaceutical compositions of the present disclosure also include those that also contain one or more other active ingredients, in addition to a compound and/or pharmaceutically acceptable salt of the present disclosure. The weight ratio of the compound and/or pharmaceutically acceptable salt of the present disclosure to the second active ingredient may be varied and will depend upon the effective dose of each ingredient. Generally, an effective dose of each will be used.

### Specific Abbreviations Used

| | |
|---|---|
| AIDS | Acquired Immune Deficiency Syndrome |
| ATP | Adenosine Triphosphate |
| Blk | B lymphocyte kinase |
| BMX | Bone marrow-expressed kinase |
| BTK | Bruton's Tyrosine Kinase |
| DMSO | Dimethyl sulfoxide |
| EDTA | Ethylenediaminetetraacetic acid |
| FCS | Fetal Calf serum |
| HIV | Human immunodeficiency virus |
| Jak3 | Janus Kinase 3 |
| ITK | Interleukin-2 inducible T-cell kinase |
| NK/T-cell | Natural killer T-cell |
| PBMC | Peripheral blood mononuclear cells |
| PBS | Phosphate buffered saline |
| RPMI | Roswell Park Memorial Institute medium |
| RLK/TXK | Resting lymphocyte kinase |
| TEC | Tyrosine kinase expressed in carcinoma |
| TEC-family | Family of TEC-protein-tyrosine kinases |
| kinase | (TEC, ITK, RLK, Bmx, BTK) |
| MS | Mass spectrometry |
| ml | Milliliter |
| μl | Microliter |
| mmol | Millimole |
| THF | Tetrahydrofuran |
| MeOH | Methanol |
| EtOH | Ethanol |
| DCM | Dichloromethane |
| DCE | Dichloroethane |
| EtOAc | Ethyl acetate |
| K₂CO₃ | Potassium carbonate |
| TEA | Triethylamine |
| DIPEA | Diisopropylethylamine |
| NaHCO₃ | Sodium bicarbonate |
| NaBH(OAc)₃ | Sodium triacetoxyborohydride |
| NaBH₄ | Sodium borohydride |
| H₂ | Hydrogen |
| TBSCl | Tert-butylchlorodimethylsilane |
| Pd/C | Palladium on carbon |
| MgSO₄ | Magnesium sulfate |
| BrCN | Cyanogen bromide |
| HCl | Hydrogen chloride |
| HATU | (1-[Bis(dimethylamino)methylene]-1H-1,2,3- |
| | triazolo[4,5-b]pyridinium 3-oxide |
| | hexafluorophosphate) |
| PG | Protecting Group |
| LG | Leaving Group |

### General Synthetic Methods

In the description of the synthetic methods described below and in the referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

The following section describes general synthetic method(s) which may be useful in the preparation of compounds of the instant invention.

Compounds of Formula I

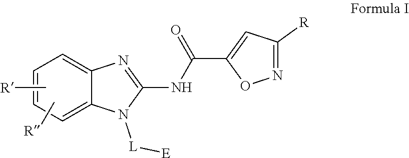

where L-E is:

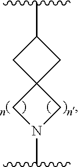

R′ is selected from —CH₂—Y wherein Y is a substituted or unsubstituted 3- to 8-membered heterocyclyl ring and R″ is hydrogen, are prepared as described below:

Reduction of intermediate Al provides intermediate A2. ProTECtion of the alcohol group with a suitable proTECtive group PG² provides intermediate A3. Reduction of the nitro group provides intermediate A4 which is then cyclized to the corresponding aminobenzimidazole intermediate A5. Coupling of intermediate A5 with an acid of formula

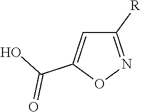

under standard coupling conditions or with an activated acid of formula

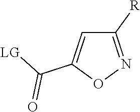

where R is as defined above and LG is a leaving group provides intermediate A6. Removal of PG² protecting group provides intermediate A7 which is oxidized to provide intermediate A8. Reductive amination of intermediate A8 with an amine of formula

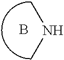

where B is a 3- to 8-membered substituted or unsubstituted heterocyclyl ring system provides intermediate A9. Removal of PG¹ protective group provides intermediate A10. Coupling of intermediate A10 with an acid of formula

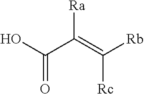

under standard coupling conditions or with an activated acid of formula

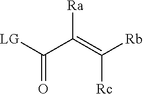

where R^{a}, R^{b} and R^{c} are as defined above and LG is a leaving group provides compounds of Formula I.

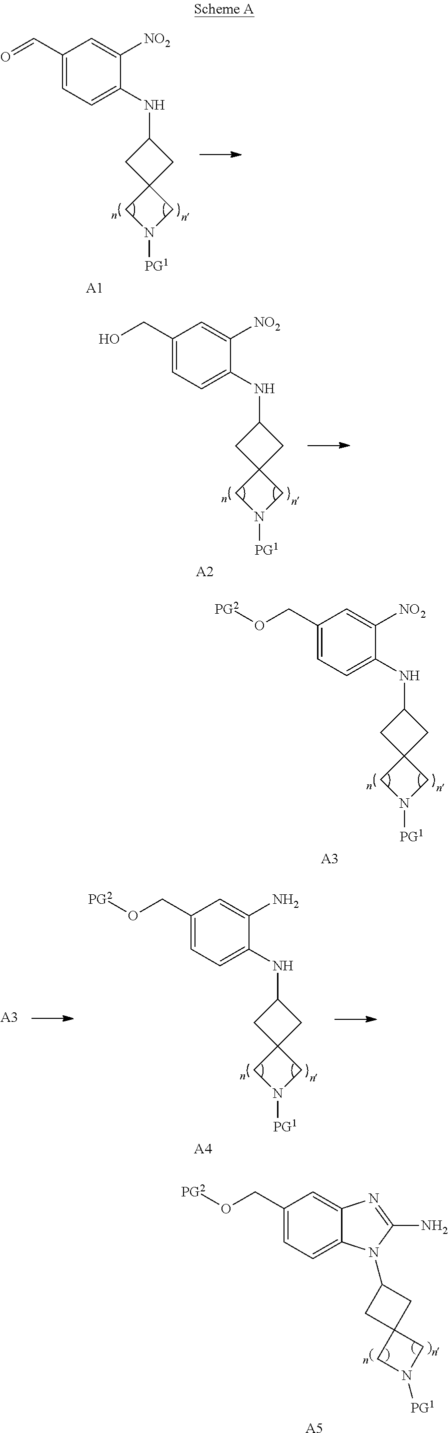

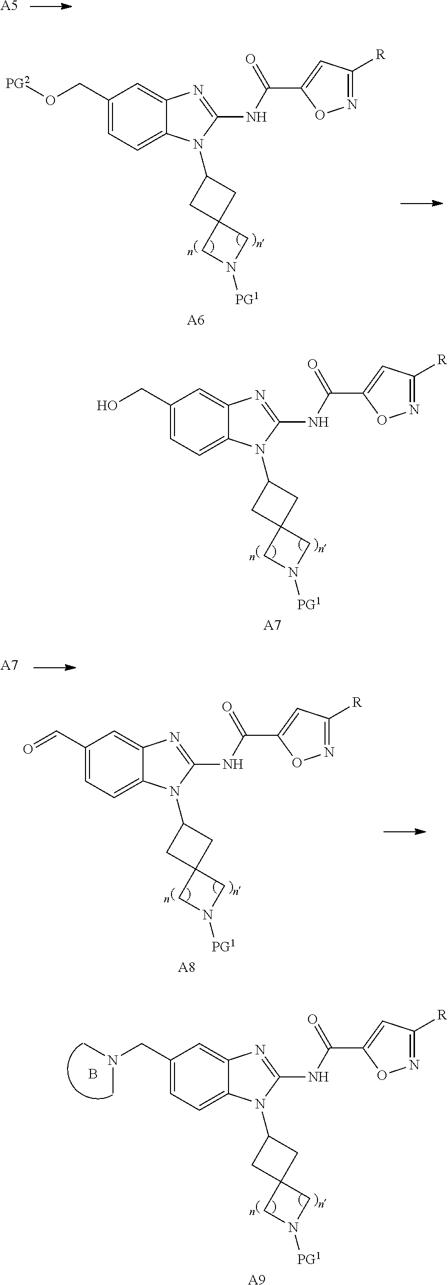

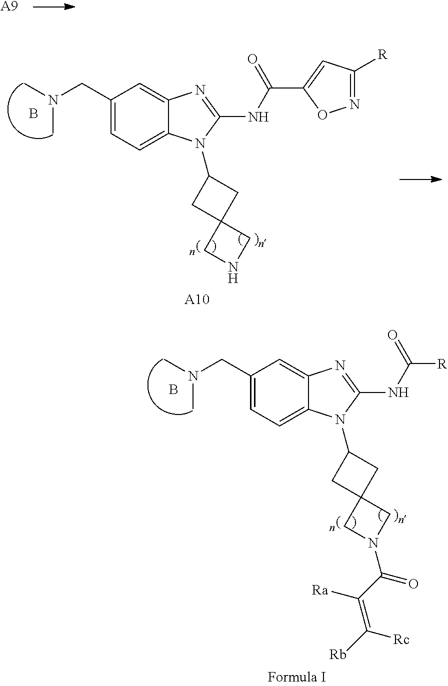

Synthesis of Intermediates 1-c and 1-c′:

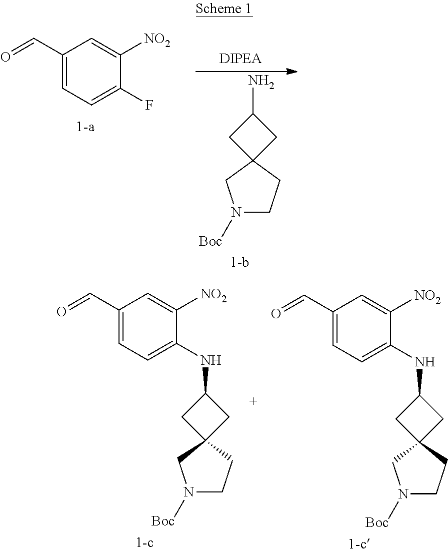 [SMILES: O=Cc1ccc(F)c([N+](=O)[O-])c1 ; CC(C)(C)OC(=O)N1CCC2(CC(N)C2)C1 ; CC(C)(C)OC(=O)N1CC[C@]2(C1)C[C@@H](Nc1ccc(C=O)cc1[N+](=O)[O-])C2 ; CC(C)(C)OC(=O)N1CC[C@]2(C1)C[C@H](Nc1ccc(C=O)cc1[N+](=O)[O-])C2]

Step 1: Intermediates 1-c and 1-c′

To a solution of 4-fluoro-3-nitrobenzaldehyde 1-a (1.0 g, 6.2 mmol) and DIPEA (3.2 ml, 18.7 mmol) in acetonitrile was added a solution of Intermediate 1-b (1.5 g, 6.5 mmol) in acetonitrile. After the addition was completed, the reaction was stirred overnight at room temperature. Volatiles were removed under reduced pressure. A saturated aqueous solution of ammonium chloride and dichloromethane were added to the residue, the organic layer was separated, and the aqueous phase was extracted twice with dichloromethane. The combined organic extracts were dried over MgSO₄, filtered and concentrated under reduced pressure. Separation by silica gel chromatography eluting with an ethyl acetate/hexane gradient provided intermediates isomers 1-c as a yellow solid and 1-c′ as a yellow solid.

### Synthesis of Intermediate 2-d:

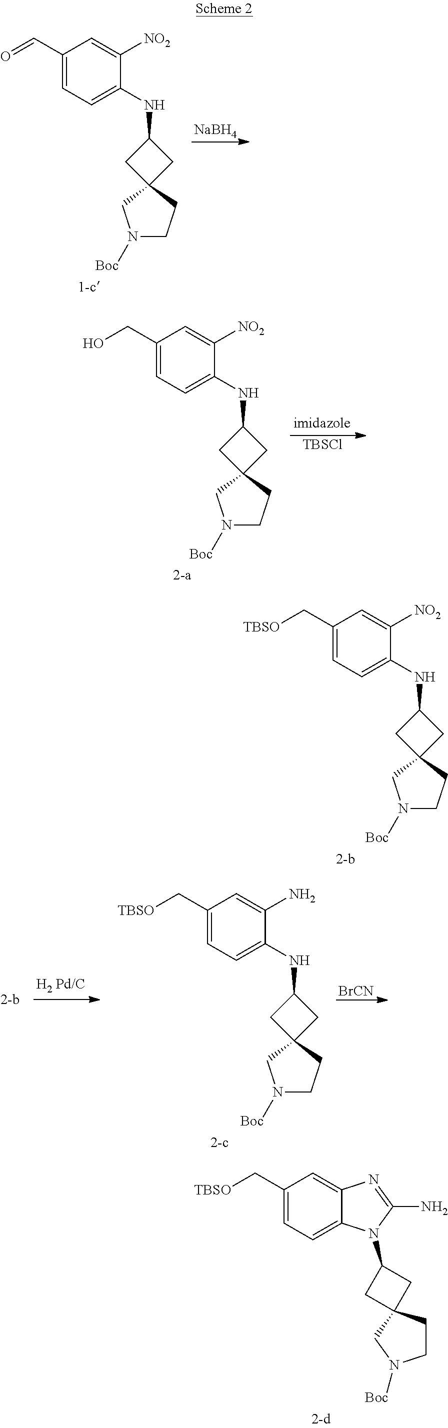

### Step 1: Intermediate 2-a

To a solution of Intermediate 1-c′ (2.0 g, 5.3 mmol) in ethanol (200 ml) was added sodium borohydride (302 mg, 8.0 mmol) and the reaction was stirred at room temperature for 1 hour. A saturated aqueous solution of NaHCO₃ was slowly added and after stirring for 15 minutes volatiles were removed under reduced pressure. Ethyl acetate was added, the organic layer was separated, washed with a saturated aqueous solution of NaHCO₃ and brine, dried over MgSO₄, filtered and concentrated under reduced pressure to provide Intermediate 2-a as a beige foam.

### Step 2: Intermediate 2-b

To a solution of Intermediate 2-a (2.0 g, 5.3 mmol) in dichloromethane (26 ml) cooled to 0° C. were sequentially added imidazole (433 mg, 6.4 mmol) and tert-butylchlorodimethylsilane (879 mg, 5.8 mmol). The reaction was then warmed to room temperature and stirred overnight. A saturated aqueous solution of ammonium chloride and ethyl acetate were added, the organic layer was separated, washed with a saturated aqueous solution of ammonium chloride and brine, dried over MgSO₄, filtered and concentrated under reduced pressure to provide Intermediate 2-b as beige oil.

### Step 3: Intermediate 2-c

To a solution of Intermediate 2-b (2.6 g, 5.3 mmol) in methanol and stirred under nitrogen was added 10% Pd/C (1.1 g, 0.6 mmol). The reaction mixture was purged with H2 and stirred for 24 hours under H2. The reaction was then filtered through celite and the filtrate was concentrated under reduced pressure to provide Intermediate 2-c as a beige solid.

### Step 4: Intermediate 2-d

To a solution of Intermediate 2-c (850 mg, 1.8 mmol) in EtOH (19 ml) was added cyanogen bromide (244 mg, 2.3 mmol) and the reaction was stirred overnight at room temperature. Volatiles were removed under reduced pressure. A saturated aqueous solution of sodium bicarbonate and ethyl acetate were then added to the residue, the organic layer was separated, washed with a saturated aqueous solution of NaHCO3 and brine, dried over MgSO4, filtered and concentrated under reduced pressure to provide Intermediate 2-d as a beige solid.

### Synthesis of Intermediate 3-d:

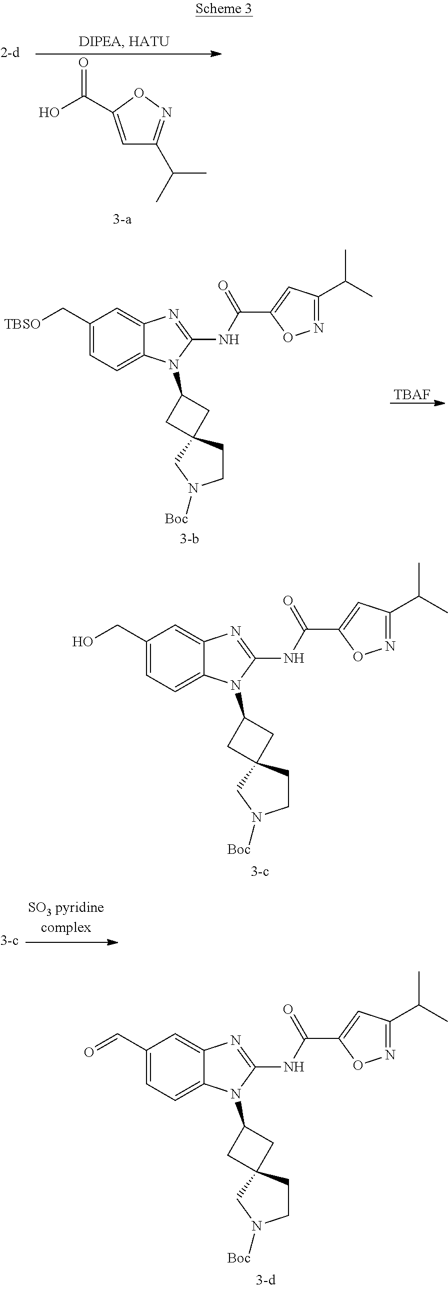 [SMILES: CC(C)c1cc(C(=O)O)on1 ; CC(C)c1cc(C(=O)Nc2nc3cc(CO[Si](C)(C)C(C)(C)C)ccc3n2[C@H]2C[C@@]3(CCN(C(=O)OC(C)(C)C)C3)C2)on1 ; "TBAF" ; CC(C)c1cc(C(=O)Nc2nc3cc(CO)ccc3n2[C@H]2C[C@@]3(CCN(C(=O)OC(C)(C)C)C3)C2)on1 ; CC(C)c1cc(C(=O)Nc2nc3cc(C=O)ccc3n2[C@H]2C[C@@]3(CCN(C(=O)OC(C)(C)C)C3)C2)on1]

### Step 1: Intermediate 3-b

To a solution of 3-isopropylisoxazole-5-carboxylic acid 3-a (591 mg, 3.1 mmol) in DMF (5.0 ml) cooled to 0° C. was added HATU (1.2 g, 3.1 mmol) and after stirring for 30 minutes a solution of intermediate 2-d (1.0 g, 2.0 mmol) and DIPEA (1.1 ml, 6.2 mmol) in DMF was added. The reaction was then stirred at room temperature overnight. A saturated aqueous solution of ammonium chloride and ethyl acetate were then added, the organic layer was separated, washed with a saturated aqueous solution of NaHCO3 and brine, dried over MgSO4, filtered and concentrated under reduced pressure. Purification by silica gel chromatography provided Intermediate 3-b as a beige solid.

### Step 2: Intermediate 3-c

To a solution of Intermediate 3-b (875 mg, 1.4 mmol) in DCM (10.0 ml) was added a 1.0 M solution of TBAF in THF (1.5 ml, 1.5 mmol) and the solution was stirred at room temperature for 30 minutes. A saturated aqueous solution of ammonium chloride and ethyl acetate were then added, the organic layer was separated, washed with brine, dried over MgSO4, filtered and concentrated under reduced pressure. Purification by silica gel chromatography provided Intermediate 3-c as a beige solid.

### Step 3: Intermediate 3-d

To a solution of Intermediate 3-c (540 mg, 1.1 mmol) in THF (5.3 ml) and DMSO (5.3 ml) cooled to 0° C. were sequentially added DIPEA (734 μl, 4.2 mmol) and a solution of SO3 pyridine complex (506 mg, 3.2 mmol) in DMSO (1.0 mL). The mixture was stirred at 0° C. until completion. Volatiles were removed under reduced pressure, water was added, a precipitate formed and was collected by filtration, washed with water and dried under vacuum to provide Intermediate 3-d as a beige solid.

### Synthesis of Compound 4:

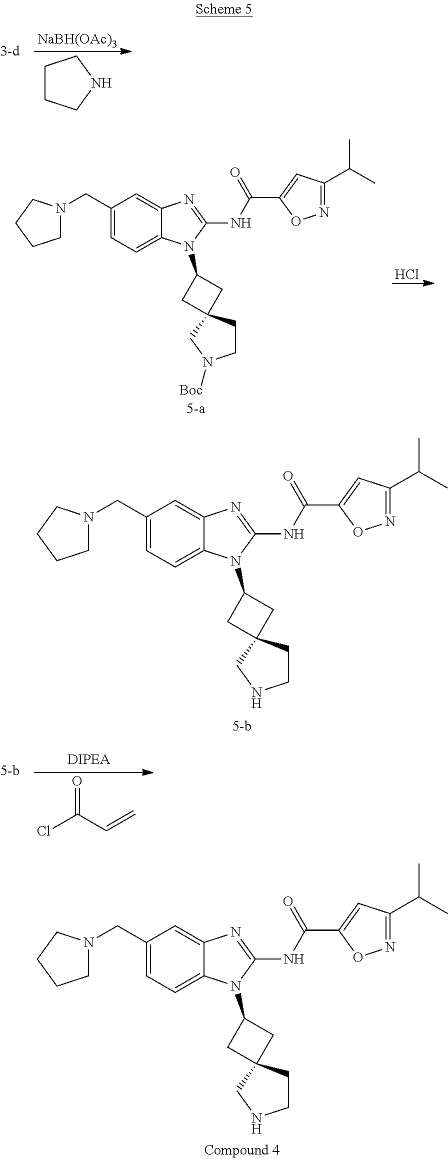

### Step 1: Intermediate 5-a

To a solution of Intermediate 3-d (474 mg, 0.9 mmol) and pyrrolidine (85 μl, 1.0 mmol) in DCE (10.0 ml) was added sodium triacetoxyborohydride (297 mg, 1.4 mmol) and the reaction was stirred at room temperature overnight. A saturated aqueous solution of NaHCO3 and ethyl acetate were then added, the organic layer was separated, washed with brine, dried over MgSO4, filtered and concentrated under reduced pressure to provide intermediate 5-a as a white solid.

### Step 2: Intermediate 5-b

To a solution of intermediate 5-a (480 mg, 0.8 mmol) in MeOH (500 μl) was added a solution of 4 N HCl in 1,4-dioxane (2.0 ml) and the reaction was stirred for 1 hour at room temperature. Volatiles were removed under reduced pressure. Diethyl ether was added, a precipitate formed and was collected by filtration, dried under vacuum to provide Intermediate 5-b.2HCl as a white solid.

### Step 3: Compound 4

To a solution of Intermediate 5-b.2HCl (1.1 g, 2.0 mmol) in tetrahydrofuran (20.0 ml) cooled to −78° C. were sequentially added DIPEA (3.0 ml, 17.2 mmol) and acryloyl chloride (180 μl, 2.2 mmol) and the reaction was stirred at −78° C. for 15 minutes. Water (20 mL) and ethyl acetate (20 mL) were added; the organic layer was separated, washed with a saturated aqueous solution of NaHCO3 and brine, dried over anhydrous MgSO4, filtered and concentrated under reduced pressure. Purification by reverse phase chromatography provided Compound 4 as a white solid.

Compounds 1, 5, 6, 7 and 8 were prepared starting from Intermediate 2-d, in a similar manner to Compound 4, by replacing

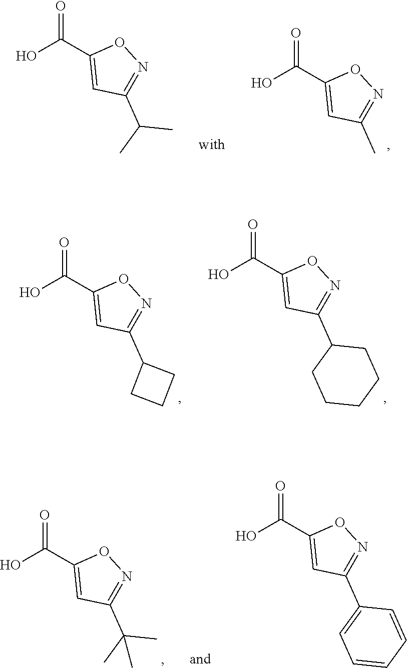

respectively for the synthesis of Intermediate 3-b.

Compounds 2 and 3 were prepared, in a similar manner to Compound 1, by replacing

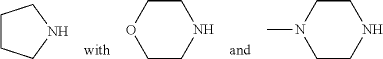

respectively.

TABLE 1
EXAMPLES OF COMPOUNDS OF FORMULA I

| Compound | Structure | MS (m/z) |
|---|---|---|
| 1 | 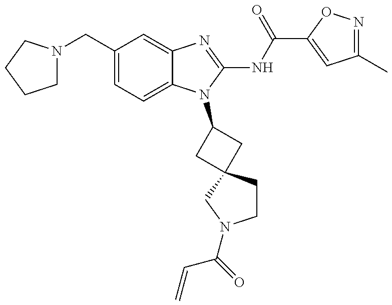 [SMILES: C=CC(=O)N1CC[C@]2(C1)C[C@H](n1c(NC(=O)c3cc(C)no3)nc3cc(CN4CCCC4)ccc31)C2] | [M + H]⁺ = 489.0 |
| 2 | 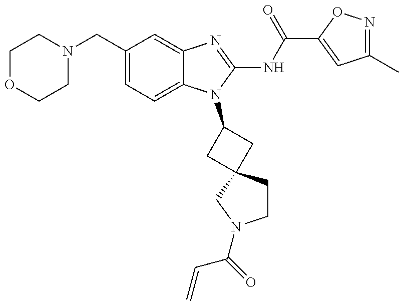 [SMILES: C=CC(=O)N1CC[C@]2(C1)C[C@H](n1c(NC(=O)c3cc(C)no3)nc3cc(CN4CCOCC4)ccc31)C2] | [M + H]⁺ = 505.0 |
| 3 | 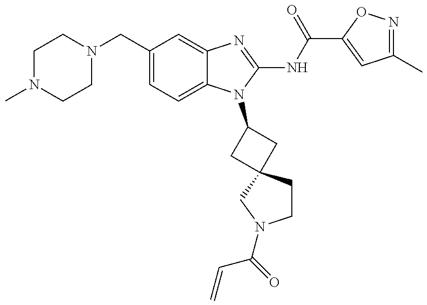 | [M + H]⁺ = 518.6 |
| 4 | 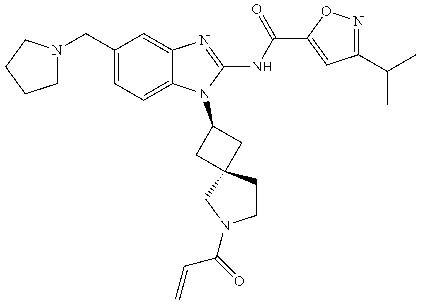 [SMILES: C=CC(=O)N1CC[C@]2(C1)C[C@H](n1c(NC(=O)c3cc(C(C)C)no3)nc3cc(CN4CCCC4)ccc31)C2] | [M + H]⁺ = 517.4 |
| 5 | 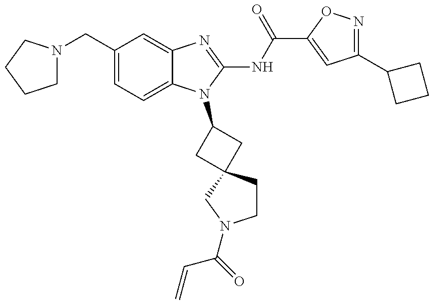 [SMILES: C=CC(=O)N1CC[C@]2(C1)C[C@H](n1c(NC(=O)c3cc(C4CCC4)no3)nc3cc(CN4CCCC4)ccc31)C2] | [M + H]⁺ = 529.0 |
| 6 | 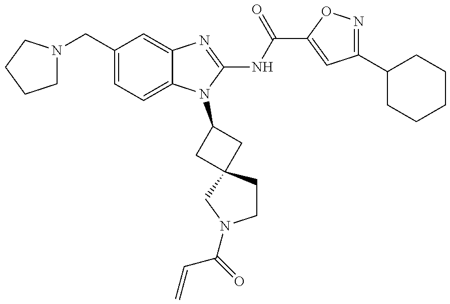 [SMILES: C=CC(=O)N1CC[C@]2(C1)C[C@H](n1c(NC(=O)c3cc(C4CCCCC4)no3)nc3cc(CN4CCCC4)ccc31)C2] | [M + H]⁺ = 557.0 |
| 7 | 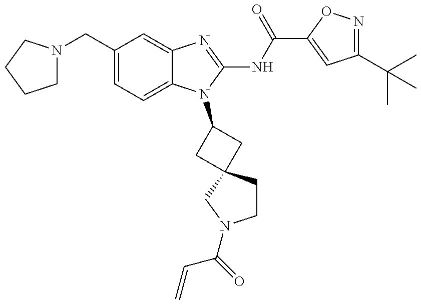 [SMILES: C=CC(=O)N1CC[C@]2(C1)C[C@H](n1c(NC(=O)c3cc(C(C)(C)C)no3)nc3cc(CN4CCCC4)ccc31)C2] | [M + H]⁺ = 532.0 |
| 8 | 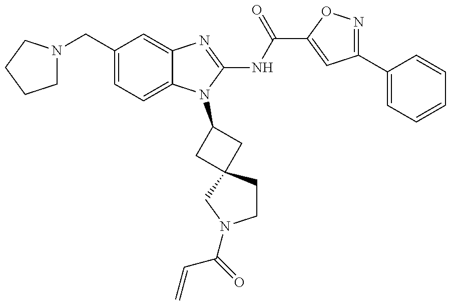 [SMILES: C=CC(=O)N1CC[C@]2(C1)C[C@H](n1c(NC(=O)c3cc(-c4ccccc4)no3)nc3cc(CN4CCCC4)ccc31)C2] | [M + H]⁺ = 551.0 |

### Example 1

### Biochemical Kinase Assays

The in vitro kinase assays were performed at Nanosyn (Santa Clara, Calif.) utilizing micro-fluidic detection Technology. The test compounds were serially pre-diluted in DMSO and added, by the acoustic dispensing (Labcyte® 550), directly to 384we11 assay plates into 10 uL of a buffer with enzyme comprising: 100 mM HEPES, pH7.5, 5 mM MgCl2, 0.1% bovine serum albumin, 1 mM DTT, 0.01% Triton X-100 and the enzyme. Final DMSO concentration was maintained at 1% in all samples, including the controls. The reactions were initiated by addition of ATP (to the specified concentration) and the fluorescently labeled peptide substrate to a final concentration of 1 uM, and incubated for 3 hours at 250 C. Following incubation, the reactions were quenched by addition of 40 μL of termination buffer (100 mM HEPES, pH7.5, 0.01% Triton X-100, 50 mM EDTA). Terminated plates were analyzed using Caliper LabChip® 3000 microfluidic electrophoresis instrument (Caliper Life Sciences/Perkin Elmer). The enzymatic modification of the peptide substrate (phosphorylation) results in a change of net charge enabling electrophoretic separation of product from substrate. As substrate and product are separated by electrophoresis, two peaks of fluorescence are observed. Change in the relative fluorescence intensity of the substrate and product peaks was the parameter measured, reflecting enzyme activity. In the presence of inhibitor, the ratio between product and substrate is altered: signal of the product decreases, while the signal of the substrate increases. Activity in each test sample was determined as the product to sum ratio (PSR): P/(S+P), where P is the peak height of the product and S is the peak height of the FAM-cAMP substrate. For each compound, enzyme activity was measured at 12 concentrations spaced by 3× dilution intervals. Negative control samples (0%−inhibition in the absence of inhibitor, DMSO only) and positive control samples (100%−inhibition, in the absence of enzyme or in the presence of control inhibitor) were assembled in replicates of four and were used to calculate %−inhibition values in the presence of compounds. Percent inhibition (Pinh) was determined using the following equation: Pinh=(PSR0%−PSRinh)/(PSR0%−PSR100%)*100, where PSRinh is the product sum ratio in the presence of inhibitor, PSR0% is the product sum ratio in the absence of inhibitor and PSR100% is the product sum ratio in 100%-inhibition control samples. To determine IC50 values, the inhibition curves (Pinh versus inhibitor concentration) were fitted by 4 parameter sigmoid dose-response model using XLfit software (IDBS).

TABLE 2
RESULTS OF BIOCHEMICAL KINASE INHIBITION ASSAY

| Compound | ITK | TXK | BTK | TEC | ITK/TXK |
|---|---|---|---|---|---|
| Number | IC50 (nM) | IC50 (nM) | IC50 (nM) | IC50 (nM) | Ratio |
| 1 | 127 | 5.28 | 16.1 | 95.5 | 24.1 |
| 2 | 40.9 | 3.02 | 14.7 | 11.1 | 13.5 |
| 3 | 80.9 | 3.48 | 17.9 | 43.9 | 23.2 |
| 4 | 553 | 1.42 | 12.0 | 96.3 | 390 |
| 5 | 576 | 1.25 | 7.65 | 50.5 | 461 |
| 6 | >1000 | 1.78 | 10.1 | 105 | >561 |
| 7 | 536 | 5.04 | 59.7 | 529 | 106 |
| 8 | 369 | 1.97 | 25.7 | 136 | 187 |

Data indicate that the compounds of the instant invention are highly specific for TXK relative to other kinases, especially ITK, which is expected to provide advantages in safety and tolerability.

The various embodiments described above can be combined to provide further embodiments. All of the patents, patent application publications, patent applications, foreign patents, foreign patent applications and non-patent publications referred to in this specification are incorporated herein by reference, in their entirety. Aspects of the embodiments can be modified, if necessary to employ concepts of the various patents, applications and publications to provide yet further embodiments.

The disclosure of Canadian patent application Ser. No. 2,976,819, filed Aug. 21, 2017, is incorporated herein in its entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the structure of Formula I: 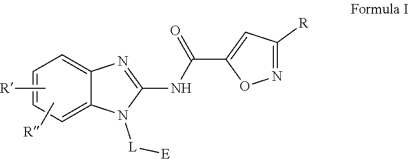
or pharmaceutically acceptable salt, solvate, solvate of salt, stereoisomer, tautomer, isotope, prodrug, complex or biologically active metabolite thereof; wherein
R is substituted or unsubstituted alkyl, substituted or unsubstituted alkenyl, substituted or unsubstituted alkynyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl;
L is: 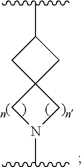
wherein
n is an integer from 1 to 3;
n′ is an integer from 1 to 3;
E is: 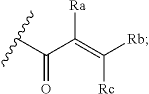
wherein
Ra, Rb, and Rc are each, independently, hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a 3- to 8-membered substituted or unsubstituted cycloalkyl ring or form a 3- to 8-membered substituted or unsubstituted heterocyclic ring, and Rc is hydrogen, halogen, —CN, substituted or unsubstituted alkyl, substituted or unsubstituted heteroalkyl, substituted or unsubstituted cycloalkyl, or substituted or unsubstituted heterocyclyl; or
Rb and Rc, taken together with the carbon atom to which they are attached, form a 3- to 8-membered substituted or unsubstituted cycloalkyl ring or form a 3- to 8-membered heterocyclic ring, and Ra is hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a triple bond, and Rc is hydrogen, halogen, —CN, substituted or unsubstituted alkyl, substituted or unsubstituted heteroalkyl, substituted or unsubstituted cycloalkyl, or substituted or unsubstituted heterocyclyl; and
R′ and R″ are each, independently, —X—Y; wherein
X is alkylene, -(alkylene)—NR¹—, -(alkylene)—NR²—, -(alkylene)—O—, —O—, —S—, —S(O)ₘ—, —NR¹—, —NR²—, —C(O)—, —C(O)O—, —C(O)NR¹—, —C(O)ONR¹—, or —S(O)ₘNR¹—;
R¹ is selected from hydrogen, lower alkyl or lower cycloalkyl;
R² is selected from —C(O)R³, —C(O)OR³ or —S(O)ₘR³;
R³ is selected from lower alkyl or lower cycloalkyl;
m is an integer from 1 to 2; or
X is a bond; and
Y is hydrogen, halogen, substituted or unsubstituted alkyl, substituted or unsubstituted alkenyl, substituted or unsubstituted alkynyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl; or
R′ and R″ taken together with the carbon atoms to which they are attached form a 3- to 8-membered substituted or unsubstituted cycloalkyl ring, or a 3- to 8-membered substituted or unsubstituted heterocyclyl ring.

2. The compound according to claim 1, or pharmaceutically acceptable salt, solvate, solvate of salt, stereoisomer, tautomer, isotope, prodrug, complex or biologically active metabolite thereof, wherein -L-E is: 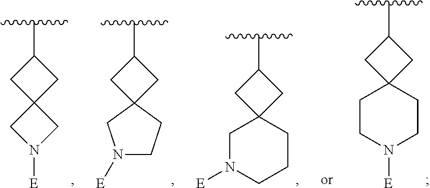
and
E is: 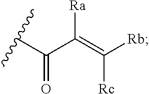
wherein
Ra, Rb, and Rc are each, independently, hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a 3- to 8-membered substituted or unsubstituted cycloalkyl ring or form a 3- to 8-membered substituted or unsubstituted heterocyclic ring, and Rc is hydrogen, halogen, —CN, substituted or unsubstituted alkyl, substituted or unsubstituted heteroalkyl, substituted or unsubstituted cycloalkyl, or substituted or unsubstituted heterocyclyl; or
Rb and Rc, taken together with the carbon atom to which they are attached, form a 3- to 8-membered substituted or unsubstituted cycloalkyl ring or form a 3- to 8- membered heterocyclic ring, and Ra is hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a triple bond, and Rc is hydrogen, halogen, —CN, substituted or unsubstituted alkyl, substituted or unsubstituted heteroalkyl, substituted or unsubstituted cycloalkyl, or substituted or unsubstituted heterocyclyl.

3. The compound according to claim 1, or pharmaceutically acceptable salt, solvate, solvate of salt, stereoisomer, tautomer, isotope, prodrug, complex or biologically active metabolite thereof, wherein -L-E is: 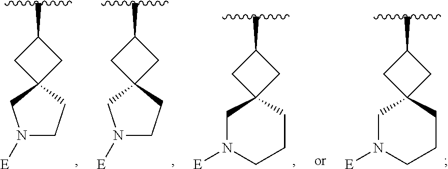
and
E is: 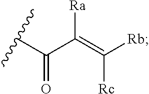
wherein
Ra, Rb, and Rc are each, independently, hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a 3- to 8-membered substituted or unsubstituted cycloalkyl ring or form a 3- to 8-membered substituted or unsubstituted heterocyclic ring, and Rc is hydrogen, halogen, —CN, substituted or unsubstituted alkyl, substituted or unsubstituted heteroalkyl, substituted or unsubstituted cycloalkyl, or substituted or unsubstituted heterocyclyl; or
Rb and Rc, taken together with the carbon atom to which they are attached, form a 3- to 8-membered substituted or unsubstituted cycloalkyl ring or form a 3- to 8-membered heterocyclic ring, and Ra is hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a triple bond, and Rc is hydrogen, halogen, —CN, substituted or unsubstituted alkyl, substituted or unsubstituted heteroalkyl, substituted or unsubstituted cycloalkyl, or substituted or unsubstituted heterocyclyl.

4. The compound according to claim 1, or pharmaceutically acceptable salt, solvate, solvate of salt, stereoisomer, tautomer, isotope, prodrug, complex or biologically active metabolite thereof, wherein -L-E is: 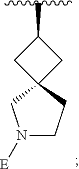
and
E is: 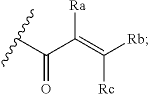
wherein
Ra, Rb, and Rc are each, independently, hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a 3- to 8-membered substituted or unsubstituted cycloalkyl ring or form a 3- to 8-membered substituted or unsubstituted heterocyclic ring, and Rc is hydrogen, halogen, —CN, substituted or unsubstituted alkyl, substituted or unsubstituted heteroalkyl, substituted or unsubstituted cycloalkyl, or substituted or unsubstituted heterocyclyl; or
Rb and Rc, taken together with the carbon atom to which they are attached, form a 3- to 8-membered substituted or unsubstituted cycloalkyl ring or form a 3- to 8-membered heterocyclic ring, and Ra is hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb, taken together with the carbon atoms to which they are attached, form a triple bond, and Rc is hydrogen, halogen, —CN, substituted or unsubstituted alkyl, substituted or unsubstituted heteroalkyl, substituted or unsubstituted cycloalkyl, or substituted or unsubstituted heterocyclyl.

5. The compound according to any one of claims 2 to 4, or pharmaceutically acceptable salt, solvate, solvate of salt, stereoisomer, tautomer, isotope, prodrug, complex or biologically active metabolite thereof, wherein E is:

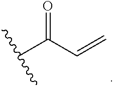 [SMILES: *C(=O)C=C]

6. The compound according to claim 1, or pharmaceutically acceptable salt, solvate, solvate of salt, stereoisomer, tautomer, isotope, prodrug, complex or biologically active metabolite thereof, wherein R′ is —CH₂—Y and R″ is hydrogen.

7. The compound according to claim 6, or pharmaceutically acceptable salt, solvate, solvate of salt, stereoisomer, tautomer, isotope, prodrug, complex or biologically active metabolite thereof, wherein Y is substituted or unsubstituted heterocyclyl.

8. A compound having the structure of Formula II: 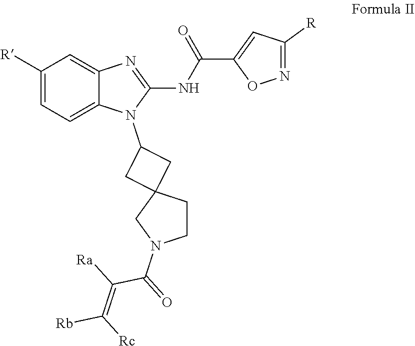
or a pharmaceutically acceptable salt, solvate, solvate of salt, isotope, prodrug, complex, or biologically active metabolite thereof; wherein
R is substituted or unsubstituted alkyl, substituted or unsubstituted cycloalkyl, substituted or unsubstituted heterocyclyl, substituted or unsubstituted aryl, or substituted or unsubstituted heteroaryl;
Ra, Rb, and Rc are each, independently, hydrogen, halogen, —CN, substituted or unsubstituted alkyl, substituted or unsubstituted heteroalkyl, substituted or unsubstituted cycloalkyl, or substituted or unsubstituted heterocyclyl; and
R′ is —X—Y; wherein
X is a bond or alkylene; and
Y is hydrogen, substituted or unsubstituted cycloalkyl, substituted or unsubstituted heterocyclyl, substituted or unsubstituted aryl, or substituted or unsubstituted heteroaryl.

9. A compound having the structure:

| Compound | Structure |
|---|---|
| 1 | 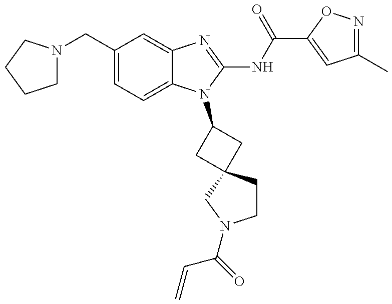 [SMILES: C=CC(=O)N1CC[C@]2(C1)C[C@H](n1c(NC(=O)c3cc(C)no3)nc3cc(CN4CCCC4)ccc31)C2] |
| 2 | 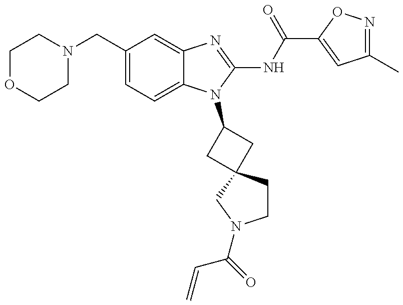 [SMILES: C=CC(=O)N1CC[C@]2(C1)C[C@H](n1c(NC(=O)c3cc(C)no3)nc3cc(CN4CCOCC4)ccc31)C2] |
| 3 | 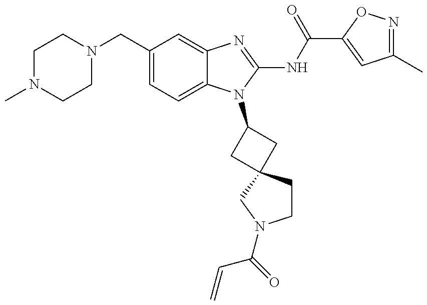 |
| 4 | 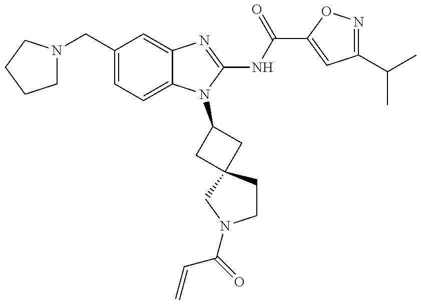 [SMILES: C=CC(=O)N1CC[C@]2(C1)C[C@H](n1c(NC(=O)c3cc(C(C)C)no3)nc3cc(CN4CCCC4)ccc31)C2] |
| 5 | 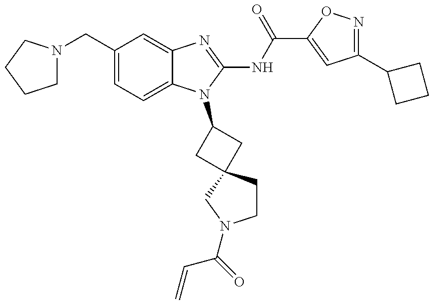 [SMILES: C=CC(=O)N1CC[C@]2(C1)C[C@H](n1c(NC(=O)c3cc(C4CCC4)no3)nc3cc(CN4CCCC4)ccc31)C2] |
| 6 | 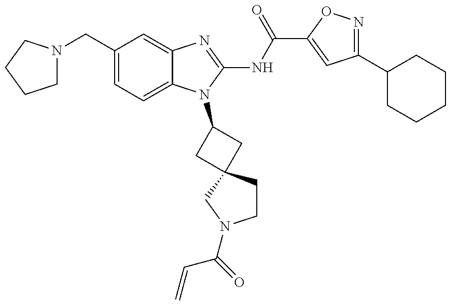 [SMILES: C=CC(=O)N1CC[C@]2(C1)C[C@H](n1c(NC(=O)c3cc(C4CCCCC4)no3)nc3cc(CN4CCCC4)ccc31)C2] |
| 7 | 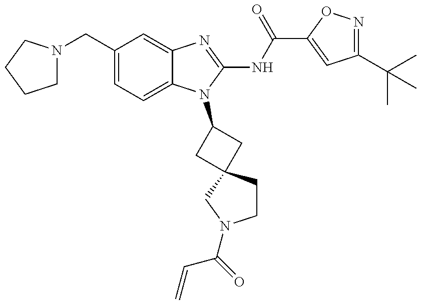 [SMILES: C=CC(=O)N1CC[C@]2(C1)C[C@H](n1c(NC(=O)c3cc(C(C)(C)C)no3)nc3cc(CN4CCCC4)ccc31)C2] |
| 8 | 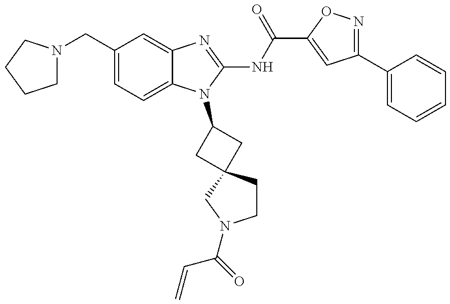 [SMILES: C=CC(=O)N1CC[C@]2(C1)C[C@H](n1c(NC(=O)c3cc(-c4ccccc4)no3)nc3cc(CN4CCCC4)ccc31)C2] |

or pharmaceutically acceptable salt, solvate, solvate of salt, stereoisomer, tautomer, isotope, prodrug, complex or biologically active metabolite thereof.

10. The compound of any one of claims 1 to 9, or pharmaceutically acceptable salt, solvate, solvate of salt, stereoisomer, tautomer, isotope, prodrug, complex or biologically active metabolite thereof, for use in therapy.

11. The compound of any one of claims 1 to 9, or pharmaceutically acceptable salt, solvate, solvate of salt, stereoisomer, tautomer, isotope, prodrug, complex or biologically active metabolite thereof, for use in preventing or treating cancer, autoimmune diseases, allergic diseases, neurological disease, inflammatory diseases and/or viral infection.

12. The compound of any one of claims 1 to 9, or pharmaceutically acceptable salt, solvate, solvate of salt, stereoisomer, tautomer, isotope, prodrug, complex or biologically active metabolite thereof, for use in the preparation of a medicament for inhibiting a protein kinase in a subject.

13. A pharmaceutical composition comprising the compound of any one of claims 1 to 9, or pharmaceutically acceptable salt, solvate, solvate of salt, stereoisomer, tautomer, isotope, prodrug, complex or biologically active metabolite thereof, and at least one pharmaceutically acceptable carrier or excipient.

14. The pharmaceutical composition according to claim 13 for use in a treatment of a subject suffering from a disease, disorder or condition in which protein kinase activity is implicated.

15. The pharmaceutical composition according to claim 14, wherein a protein kinase mediated disease, disorder or condition is one in which a TEC-kinase-family member activity is implicated.

16. The pharmaceutical composition according to claim 15, wherein a protein kinase mediated disease, disorder, or condition is one in which ITK, TXK, TEC, BTK or combinations thereof are implicated.

17. A pharmaceutical composition comprising the compound of any one of claims 1 to 9, or pharmaceutically acceptable salt, solvate, solvate of salt, stereoisomer, tautomer, isotope, prodrug, complex or biologically active metabolite thereof, with at least one pharmaceutically acceptable carrier or excipient for use in therapy.

18. The pharmaceutical composition of claim 17 comprising the compound of any one of claims 1 to 9 for use in treatment with at least one additional active pharmaceutical ingredient for the treatment of cancer, autoimmune diseases, allergic diseases, inflammatory diseases, neurological diseases or viral infection wherein: said additional active pharmaceutical ingredient is appropriate for the disease being treated; and said additional active pharmaceutical ingredient is administered together with said composition as a single dosage form or separately from said composition as part of a multiple dosage form.

19. The pharmaceutical composition of claim 18, wherein the pharmaceutical compositions is a pharmaceutical form suitable for oral administration;
parenteral form; a drop infusion preparations; an inhalation, an eye lotion, a topical administration, an ointment; or a suppository.

20. A method for treating a subject suffering from a protein kinase mediated disease, disorder or condition mediated by a protein kinase, the method comprising administering to a subject in need thereof the compound of any one of claims 1 to 9 or pharmaceutical composition of claim 13, or a pharmaceutically acceptable salt solvate, solvate of a salt, stereoisomer, tautomer, isotope, prodrug, complex or biologically active metabolite thereof.

21. The method of claim 20, wherein the disease, disorder or condition is associated with the activity of a kinase member of the TEC-kinase-family.

22. The method of claim 21, wherein the disease, disorder or condition is associated with the activity of a kinase selected from ITK, TXK, BTK, TEC or a combination thereof.

23. The method of claim 21, wherein the disease, disorder or condition is associated with the activity of TXK kinase.

24. The method of claim any one of claims 20 to 23, wherein the pharmaceutical compositions may be in a conventional pharmaceutical form suitable for oral administration selected from tablets, capsules, granules, powders and syrups; parenteral administration as: intravenous, intramuscular, or subcutaneous injections; drop infusion preparations, inhalation, eye lotion, topical administration, ointment, or suppositories.

25. The method of any one of claims 20 to 24 further comprising administering to a subject suffering from a protein kinase mediated disease or condition one or more additional active pharmaceutical ingredients for the treatment of disease, disorder or condition associated with TEC-kinase family members.

26. A method of modulating kinase activity in a subject comprising administering a therapeutically effective amount of the compound of any one of claims 1 to 9 or pharmaceutical composition of claim 13, or a pharmaceutically acceptable salt solvate, solvate of a salt, stereoisomer, tautomer, isotope, prodrug, complex or biologically active metabolite thereof.

27. A method of inhibiting protein kinase in a cell or tissue comprising contacting the cell or tissue with the compound of any one of claims 1 to 9 or pharmaceutical composition of claim 13, or a pharmaceutically acceptable salt solvate, solvate of a salt, stereoisomer, tautomer, isotope, prodrug, complex or biologically active metabolite thereof.

28. A method of inhibiting protein kinase activity in a subject, comprising administering a therapeutically effective amount of the compound of any one of claims 1 to 9 or pharmaceutical composition of claim 13, or a pharmaceutically acceptable salt solvate, solvate of a salt, stereoisomer, tautomer, isotope, prodrug, complex or biologically active metabolite thereof.

29. The method according to any one of claims 26 to 28, wherein said target kinase activity is associated with TEC-kinase-family.

30. The method according to claim 29, wherein said target kinase is ITK, TXK, BTK, or TEC, or combinations thereof.

31. The method according to claim 30, wherein said target kinase is TXK.

32. A method of treating a subject suffering from cancer, autoimmune diseases, allergic diseases, inflammatory diseases, neurological diseases, viral infection or combinations thereof, comprising administering to the subject a therapeutically effective amount of the compound of any one of claims 1 to 9 or pharmaceutical composition of claim 13, or a pharmaceutically acceptable salt solvate, solvate of a salt, stereoisomer, tautomer, isotope, prodrug, complex or biologically active metabolite thereof, wherein the enzymatic activity of TXK, ITK, BTK, TEC or a combination thereof are reduced.

33. The method according to claim 32, wherein the disease is lung inflammation, allergic asthma, pneumonia, psoriasis, atopic dermatitis, uveitis, dry eye disease, arthritis, systemic lupus erythematosus, rheumatoid arthritis, psoriatic arthritis, Still's disease, juvenile arthritis, type I diabetes, inflammatory bowel disease, myasthenia gravis, Hashimoto's thyroiditis, Ord's thyroiditis, Basedow's disease, Sjogren's syndrome, multiple sclerosis, Guillain-Barre syndrome, acute disseminated encephalomyelitis, Addison disease, opsoclonus-myoclonus syndrome, ankylosing spondylitis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's disease, Takayasu arteritis, temporal arteritis, warm autoimmune hemolytic anemia, Wegener granuloma, alopecia universalis, Burchett disease, chronic fatigue syndrome, dysautonomia, endometriosis, interstitial cystitis, myotonia, vulvodynia, pemphigus, allergy, anaphylaxis, allergic conjunctivitis, allergic rhinitis, atopic dermatitis, asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, epicondylitis, epididymitis, fasciitis, fibrositis, gastritis, gastroenteritis, hepatitis, hidradenitis suppurativa, inflammatory bowel disease, laryngitis, mastitis, meningitis, myelitis, myocarditis, myositis nephritis, oophoritis, orchitis, osteitis, osteoarthritis, pancreatitis, parotitis, pericarditis, peritonitis, pharyngitis, pleuritis, phlebitis, pneumonia, proctitis, prostatitis, pyelonephritis, rhinitis, salpingitis, sinusitis, stomatitis, synovitis, tendinitis, tonsillitis, uveitis, vaginitis, vasculitis, vulvitis, stroke, Alzheimer's disease, Huntington's disease, amyotrophic Lateral Sclerosis, HIV/AIDS, influenza, solid tumors, non-Hodgkin's lymphoma, T-cell lymphomas, T-cell leukemias, peripheral T-cell lymphoma, Seazry syndrome/cutaneous T-cell lymphoma, acute lymphoblastic leukemia, adult T-cell leukemia/lymphoma, NK/T-cell lymphoma, nasal type or aggressive NK-cell leukemia, or combinations thereof.

34. The method according to any one of claims 20 to 33, further comprising administering a therapeutically effective amount of at least one additional active pharmaceutical ingredient for the treatment of cancer, autoimmune diseases, allergic diseases, inflammatory diseases, neurological disease and/or viral infection in combination therapy.

35. The method of claim 34, wherein the additional active pharmaceutical ingredient is a steroid, a leukotriene antagonist, a sodium channel blocker, a glutamate antagonist, an anti-histamine, an anti-cancer agent, an anti-viral agent, an anti-biotic agent, or a protein kinase inhibitor, or combinations thereof.

36. A method of modulating target kinase function comprising administering to a subject in need of such modulation the compound of any one of claims 1 to 9 or pharmaceutical composition of claim 13, or a pharmaceutically acceptable salt solvate, solvate of a salt, stereoisomer, tautomer, isotope, prodrug, complex or biologically active metabolite thereof.

37. A probe comprising the compound of any one of claims 1 to 9 which is covalently conjugated to a detectable label or affinity tag for said compound.

38. The probe according to claim 37, wherein the detectable label is a fluorescent moiety, a chemiluminescent moiety, a paramagnetic contrast agent, a metal chelate, a radioactive isotope-containing moiety, or biotin.
